(19) United States
(12) Patent Application Publication
(10) Pub. No.: US 2008/0312167 A1
(43) Pub. Date: Dec. 18, 2008

(54) 4'' Amino Linked Macrolides Useful for the Treatment of Microbial Infections

(76) Inventors: Sulejman Alihodzic, Zagreb (HR); Gorjana Lazarevski, Zagreb (HR)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/913,853

(22) PCT Filed: May 9, 2006
(86) PCT No.: PCT/IB2006/001213

§ 371 Date: Jul. 11, 2008

Related U.S. Application Data
(60) Provisional application No. 60/679,778, filed on May 10, 2005.

(51) Int. Cl. A61K 31/7048; C07H 17/08; A61P 31/04

(52) U.S. Cl. 514/28; 536/7.4

(57) ABSTRACT

The present invention relates to 15-membered macrolides substituted at the 4″ position of formula (I)

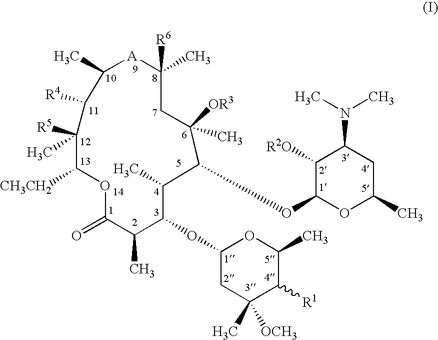

and pharmaceutically acceptable derivatives thereof, to processes for their preparation and their use in therapy or prophylaxis of systemic or topical microbial infections in a human or animal body.

The present invention claims priority to U.S. Provisional Application 60/679,778 filed May 10, 2005, herein incorporated by reference in its entirety.

### FIELD OF THE INVENTION

The present invention relates to novel semi-synthetic macrolides having antimicrobial activity, in particular antibacterial activity. More particularly, the invention relates to 15-membered macrolides substituted at the 4″ position, to processes for their preparation, to compositions containing them and to their use in medicine.

### BACKGROUND OF THE INVENTION

Macrolide antibacterial agents are known to be useful in the treatment or prevention of bacterial infections. However, the emergence of macrolide-resistant bacterial strains has resulted in the need to develop new macrolide compounds. For example, EP 0 895 999 describes derivatives modified at 4″ position which have antimicrobial activity.

### SUMMARY OF THE INVENTION

According to the present invention, we have now found novel 15-membered macrolides substituted at the 4″ position which have antimicrobial activity.

Thus, the present invention provides compounds of general formula (I)

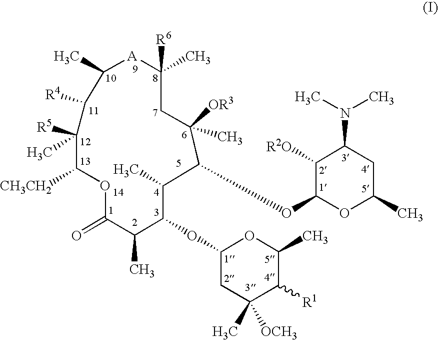

wherein
A is a bivalent radical selected from —C(O)NH—, —NHC(O)—, —N(R⁷)—CH₂— and —CH₂—N(R⁷)—;
R¹ is —NHC(O)(CH₂)_{d}XR⁸;
R² is hydrogen;
R³ is hydrogen, C₁₋₄alkyl, or C₂₋₆alkenyl optionally substituted by 9 to 10 membered fused bicyclic heteroaryl;
R⁴ is hydroxy, C₂₋₆alkenyloxy optionally substituted by 9 to 10 membered fused bicyclic heteroaryl, or C₁₋₆alkoxy optionally substituted by C₁₋₆alkoxy or —O(CH₂)ₑNR⁷R⁹,
R⁵ is hydroxy, or
R⁴ and R⁵ taken together with the intervening atoms form a cyclic group having the following structure:

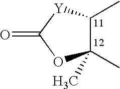

wherein Y is a bivalent radical selected from —CH₂—, —CH(CN)—, —O—, —N(R¹⁰)— and —CH(SR¹⁰)—, with proviso that when A is —NHC(O)—, —N(R⁷)—CH₂— or —CH₂—N(R⁷)—, Y is —O—;
R⁶ is hydrogen or fluorine;
R⁷ is hydrogen or C₁₋₆alkyl;
R⁸ is a heterocyclic group having the following structure:

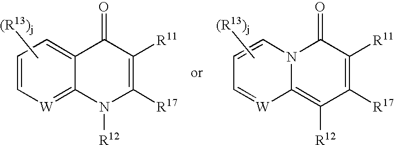

R⁹ is hydrogen or C₁₋₆alkyl;
R¹⁰ is hydrogen or C₁₋₄alkyl substituted by a group selected from optionally substituted phenyl, optionally substituted 5 or 6 membered heteroaryl and optionally substituted 9 to 10 membered fused bicyclic heteroaryl;
R¹¹ is hydrogen, —C(O)OR¹⁴, —C(O)NHR¹⁴, —C(O)CH₂NO₂ or —C(O)CH₂SO₂R⁷
R¹² is hydrogen, C₁₋₄alkyl optionally substituted by hydroxy, cyano, C₁₋₄alkoxy, NH₂, —NH(C₁₋₄alkyl) or —N(C₁₋₄alkyl)₂; C₂₋₄alkenyl optionally substituted by hydroxy, cyano, C₁₋₄alkoxy, NH₂, —NR(C₁₋₄alkyl) or —N(C₁₋₄alkyl)₂; C₁₋₄alkoxy; C₃₋₇cycloalkyl; —NH₂; —NH(C₁₋₄alkyl); —N(C₁₋₄alkyl)₂; (C₁₋₄alkyl)OC(O)N(C₁₋₄alkyl); or optionally substituted phenyl or benzyl;
R¹³ is halogen, C₁₋₄alkyl, C₁₋₄thioalkyl, C₁₋₄alkoxy, —NH₂, —NH(C₁₋₄alkyl) or —N(C₁₋₄alkyl)₂;
R¹⁴ is hydrogen or C₁₋₆alkyl optionally substituted by up to three groups independently selected from halogen, C₁₋₄alkoxy, —OC(O)C₁₋₆alkyl and —OC(O)OC₁₋₆alkyl; —(CH₂)_{q}heterocyclyl, —(CH₂)_{q}heteroaryl, —(CH₂)_{q}aryl, or —(CH₂)_{q}C₃₋₇cycloalkyl;
R¹⁵ is hydrogen, C₁₋₄alkyl, C₃₋₇cycloalkyl, optionally substituted phenyl or benzyl, acetyl or benzoyl;
R¹⁶ is hydrogen or R¹³, or R¹⁶ and R¹² are linked to form the bivalent radical —O(CH₂)₂—, —(CH₂)ₜ—, —NR⁷(CH₂)ₐ—, —OCH₂NR⁷—, —SCH₂NR⁷—, —CH₂NR⁷CH₂, —CH₂OCH₂—, —CH₂SCH₂— or
—(CH₂)ₐNR⁷—;
R¹⁷ is hydrogen, or R¹⁷ and R¹² are linked to form the bivalent radical selected from the group: —S(CH₂)_{b}—, —NR⁷(CH₂)_{b}—, and —O(CH₂)_{b}—;
R¹⁸ is C₁₋₈alkyl, C₂₋₆alkenyl or C₂₋₆alkynyl;
X is —U(CH₂)ᵥB(CH₂)ᵥD-, —U(CH₂)ᵥB—R¹⁸—, —U(CH₂)ᵥB(CH₂)ᵥD(CH₂)ᵥE-, —U(CH₂)ᵥB(CH₂)ᵥD-R¹⁸
or a group selected from:

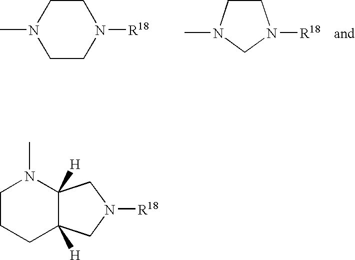

U, B, D and E are independently divalent radicals selected from —N(R¹⁵)—, —O—, —S(O)_{Z}—, —N(R¹⁵)C(O)—, —C(O)N(R¹⁵)— and —N[C(O)R¹⁵]—;

### W is —C(R¹⁶)— or —N—;

a is 1 or 2
b is an integer from 1 to 3
d is 0 or an integer from 1 to 5;
e is an integer from 2 to 4;
j and z are each independently integers from 0 to 2;
q is an integer from 0 to 4
t is 2 or 3;
v is an integer from 1 to 8;
and pharmaceutically acceptable derivatives thereof.

### DETAILED DESCRIPTION OF THE INVENTION

The term “pharmaceutically acceptable” as used herein means a compound which is suitable for pharmaceutical use. Salts and solvates of compounds of the invention which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of the invention and their pharmaceutically acceptable salts and solvates.

The term “pharmaceutically acceptable derivative” as used herein means any pharmaceutically acceptable salt, solvate or prodrug, e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives. Preferred pharmaceutically acceptable derivatives are salts, solvates, esters, carbamates and phosphate esters. Particularly preferred pharmaceutically acceptable derivatives are salts, solvates and esters. Most preferred pharmaceutically acceptable derivatives are salts and esters.

The compounds of the present invention may be in the form of and/or may be administered as a pharmaceutically acceptable salt. For a review on suitable salts see Berge et al., J. Pharm. Sci., 1977, 66, 1-19.

Typically, a pharmaceutical acceptable salt may be readily prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound of formula (I) and the resulting mixture evaporated to dryness (lyophilised) to obtain the acid addition salt as a solid. Alternatively, a compound of formula (I) may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Suitable addition salts are formed from inorganic or organic acids which form non-toxic salts and examples are hydrochloride, hydrobromide, hydroiodide, sulphate, bisulphate, nitrate, phosphate, hydrogen phosphate, acetate, trifluoroacetate, maleate, malate, fumarate, lactate, tartrate, citrate, formate, gluconate, succinate, pyruvate, oxalate, oxaloacetate, trifluoroacetate, saccharate, benzoate, alkyl or aryl sulphonates (eg methanesulphonate, ethanesulphonate, benzenesulphonate or p-toluenesulphonate) and isethionate. Typical examples include acetate and formate salts, for example the mono, di- or poli-acetate or formate salts.

Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

Compounds of the invention may have both a basic and an acidic centre may therefore be in the form of zwitterions.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention. The salts of the compound of formula (I) may form solvates (e.g. hydrates) and the invention also includes all such solvates.

The term “prodrug” as used herein means a compound which is converted within the body, e.g. by hydrolysis in the blood, into its active form that has medical effects. Pharmaceutically acceptable prodrugs are described in T. Higuchi and V. Stella, “Prodrugs as Novel Delivery Systems”, Vol. 14 of the A.C.S. Symposium Series, Edward B. Roche, ed., “Bioreversible Carriers in Drug Design”, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of structure (I). Further, in the case of a carboxylic acid (—COOH), esters may be employed, such as methyl esters, ethyl esters, and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

References hereinafter to a compound according to the invention include both compounds of formula (I) and their pharmaceutically acceptable derivatives.

With regard to stereoisomers, the compounds of structure (I) have more than one asymmetric carbon atom. In the general formula (I) as drawn, the solid wedge shaped bond indicates that the bond is above the plane of the paper. The broken bond indicates that the bond is below the plane of the paper. The wavy bond ( [symbol] ) indicates that the bond can be either above or below the plane of the paper. Thus, the present invention includes both epimers at the 4″-carbon.

It will be appreciated that the substituents on the macrolide may also have one or more asymmetric carbon atoms. Thus, the compounds of structure (I) may occur as individual enantiomers or diastereomers. All such isomeric forms are included within the present invention, including mixtures thereof.

Where a compound of the invention contains an alkenyl group, cis (Z) and trans (E) isomerism may also occur. The present invention includes the individual stereoisomers of the compound of the invention and, where appropriate, the individual tautomeric forms thereof, together with mixtures thereof.

Separation of diastereoisomers or cis and trans isomers may be achieved by conventional techniques, e.g. by fractional crystallisation, chromatography or HPLC. A stereoisomeric mixture of the agent may also be prepared from a corresponding optically pure intermediate or by resolution, such as HPLC, of the corresponding mixture using a suitable chiral support or by fractional crystallisation of the diastereoisomeric salts formed by reaction of the corresponding mixture with a suitable optically active acid or base, as appropriate.

The compounds of structure (I) may be in crystalline or amorphous form. Furthermore, some of the crystalline forms of the compounds of structure (I) may exist as polymorphs, which are included in the present invention.

R⁶ is hydrogen or fluorine. However, it will be appreciated that when A is —C(O)NH— or —CH₂—N(R⁷)—, R⁶ is hydrogen.

When R⁸ is a heterocyclic group having the following structure:

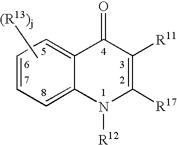

said heterocyclic is linked in the 5, 6, 7 or 8 position to the X group as above defined. In one embodiment, the heterocyclic is linked in the 6 or 7 position. In another embodiment, the heterocyclic is linked in the 5 or 8 position. When present, the R¹³ group or groups may be attached at any position on the ring. In one embodiment, an R¹³ group is attached at the 7 position.

When R⁸ is a heterocyclic group having the following structure:

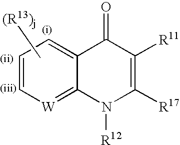

wherein W is —C(R¹⁶)— where R¹⁶ is R¹³ or R¹⁶ and R¹² are linked to form the bivalent radical —O(CH₂)₂—, —(CH₂)ₜ—, —NR⁷(CH₂)ₐ—, —OCH₂NR⁷—, —SCH₂NR⁷—, —CH₂NR⁷CH₂—, —CH₂OCH₂—, —CH₂SCH₂—, —(CH₂)ₐNR⁷—; said heterocyclic is linked in the (i), (ii) or (iii) position to the X group as above defined. In one embodiment, the heterocyclic is linked in the (i) position. In another embodiment, the heterocyclic is linked in the (ii) or (iii) position.

When R⁸ is a heterocyclic group having the following structure:

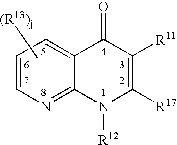

said heterocyclic is linked in the 5, 6 or 7 position to the X group as defined above. In one embodiment, the heterocyclic is linked in the 6 or 7 position. In another embodiment, the heterocyclic is linked in the 5 position.

When R⁸ is a heterocyclic group having the following structure:

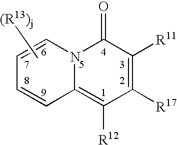

said heterocyclic is linked in the 6, 7, 8 or 9 position to the X group as above defined. In one embodiment, the heterocyclic is linked in the 7 or 8 position. In another embodiment, the heterocyclic is linked in the 6 or 9 position.

When R⁸ is a heterocyclic group having the following structure:

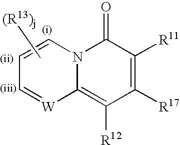

wherein W is —C(R¹⁶)— where R¹⁶ is R¹³ or R¹⁶ and R¹² are linked to form the bivalent radical —O(CH₂)₂—, —(CH₂)ₜ—, —NR⁷(CH₂)ₐ—, —OCH₂NR⁷—, —SCH₂NR⁷—, —CH₂NR⁷CH₂—, —CH₂OCH₂—, —CH₂SCH₂—, —(CH₂)ₐNR⁷—, said heterocyclic is linked in the (i), (ii) or (iii) position to the X group as above defined. In one embodiment, the heterocyclic is linked in the (i) position. In another embodiment, the heterocyclic is linked in the (ii) or (iii) position.

When R⁸ is a heterocyclic group having the following structure:

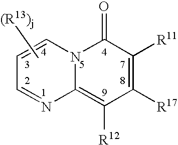

said heterocyclic is linked in the 2, 3 or 4 position to the X group as above defined. In one embodiment, the heterocyclic is linked in the 2 or 3 position. In another embodiment, the heterocyclic is linked in the 4 position.

The term “alkyl” as used herein as a group or a part of a group refers to a straight or branched hydrocarbon chain containing the specified number of carbon atoms. For example, C₁₋₁₀alkyl means a straight or branched alkyl containing at least 1, and at most 10, carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl. A C₁₋₄alkyl group is preferred, for example methyl, ethyl, n-propyl, isopropyl, n-butyl, isobutyl or t-butyl.

The term “C₃₋₇cycloalkyl” group as used herein refers to a non-aromatic monocyclic hydrocarbon ring of 3 to 7 carbon atoms such as, for example, cyclopropyl, cyclobutyl, cyclopentyl, cyclohexyl or cycloheptyl.

The term “alkoxy” as used herein refers to a straight or branched chain alkoxy group containing the specified number of carbon atoms. For example, C₁₋₆alkoxy means a straight or branched alkoxy containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy and hexyloxy. A C₁₋₄alkoxy group is preferred, for example methoxy, ethoxy, propoxy, prop-2-oxy, butoxy, but-2-oxy or 2-methylprop-2-oxy.

The term “alkenyl” as used herein as a group or a part of a group refers to a straight or branched hydrocarbon chain containing the specified number of carbon atoms and containing at least one double bond. For example, the term “C₂₋₆alkenyl” means a straight or branched alkenyl containing at least 2, and at most 6, carbon atoms and containing at least one double bond. Examples of “alkenyl” as used herein include, but are not limited to, ethenyl, 2-propenyl, 3-butenyl, 2-butenyl, 2-pentenyl, 3-pentenyl, 3-methyl-2-butenyl, 3-methylbut-2-enyl, 3-hexenyl and 1,1-dimethylbut-2-enyl. It will be appreciated that in groups of the form —O—C₂₋₆alkenyl, the double bond is preferably not adjacent to the oxygen.

The term “alkynyl” as used herein as a group or a part of a group refers to a straight or branched hydrocarbon chain containing the specified number of carbon atoms and containing at least one triple bond. For example, the term “C₂₋₆alkynyl” means a straight or branched alkynyl containing at least 2, and at most 6, carbon atoms and containing at least one triple bond. Examples of “alkynyl” as used herein include, but are not limited to, ethynyl, 2-propynyl, 3-butynyl, 2-butynyl, 2-pentynyl, 3-pentynyl, 3-methyl-2-butynyl, 3-methylbut-2-ynyl, 3-hexynyl and 1,1-dimethylbut-2-ynyl. It will be appreciated that in groups of the form —O—C₂₋₆alkynyl, the triple bond is preferably not adjacent to the oxygen.

The term “aryl” as used herein refers to an aromatic carbocyclic moiety such as phenyl, biphenyl or naphthyl.

The term “5 or 6 membered heteroaryl” as used herein as a group or a part of a group refers to a monocyclic 5 or 6 membered aromatic heterocycle containing at least one heteroatom independently selected from oxygen, nitrogen and sulfur. Examples include, but are not limited to, furanyl, thiophenyl, pyrrolyl, pyrazolyl, imidazolyl, oxazolyl, isoxazolyl, thiazolyl, isothiazolyl, triazolyl, oxadiazolyl, tetrazolyl, pyridyl, pyridazinyl, pyrazinyl, pyrimidinyl and triazinyl.

The term “9 to 10 membered fused bicyclic heteroaryl” as used herein as a group or a part of a group refers to quinolinyl, isoquinolinyl, 1,2,3,4-tetrahydroisoquinolinyl, benzofuranyl, benzimidazolyl, benzothienyl, benzoxazolyl, 1,3-benzodioxazolyl, indolyl, benzothiazolyl, furylpyridine, oxazolopyridyl or benzothiophenyl.

The term “heterocyclyl” as used herein, unless otherwise defined, refers to a monocyclic or bicyclic three- to ten-membered saturated or non-aromatic, unsaturated hydrocarbon ring containing at least one heteroatom selected from oxygen, nitrogen and sulfur. Preferably, the heterocyclyl ring has five or six ring atoms. Examples of heterocyclyl groups include, but are not limited to, pyrrolidinyl, tetrahydrofuranyl, tetrahydrothiophenyl, imidazolidinyl, pyrazolidinyl, piperidyl, piperazinyl, morpholino, tetrahydropyranyl and thiomorpholino.

The term “5 or 6 membered heterocyclic group” as used herein as a group or part of a group refers to a monocyclic 5 or 6 membered saturated hydrocarbon ring containing at least one heteroatom independently selected from oxygen, nitrogen and sulfur. Examples of such heterocyclyl groups include, but are not limited to, pyrrolidinyl, tetrahydrofuranyl, tetrahydrothiophenyl, imidazolidinyl, pyrazolidinyl, piperidyl, piperazinyl, morpholino, tetrahydropyranyl and thiomorpholino.

The term “halogen” refers to a fluorine, chlorine, bromine or iodine atom.

The terms “optionally substituted phenyl”, “optionally substituted phenyl or benzyl”, “optionally substituted 5 or 6 membered heteroaryl” or “optionally substituted 9 to 10 membered fused bicyclic heteroaryl” as used herein refer to a group which is substituted by 1 to 3 groups selected from halogen, C₁₋₄alkyl, C₁₋₄alkoxy, hydroxy, nitro, cyano, amino, C₁₋₄alkylamino or diC₁₋₄alkylamino, phenyl and 5 or 6 membered heteroaryl.

In one embodiment, A is —N(R⁷)—CH₂— or —CH₂—N(R⁷)—. A representative example of A is —N(R⁷)—CH₂—.

In one embodiment, R³ is hydrogen or C₁₋₄alkyl. A representative example of R³ is hydrogen. or methyl.

A representative example of R⁴ is hydroxy and methoxy

A representative example of R⁵ is hydroxyl.

A representative example of R⁶ is hydrogen.

A representative example of R⁷ is C₁₋₆alkyl, such as C₁₋₄alkyl, or more specifically methyl.

Representative examples of R⁸ include heterocyclic groups having the following structure:

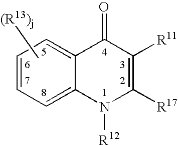

wherein the heterocyclic is linked in the 6 or 7 position to the X group as above defined. In particular, the heterocyclic is linked in the 6 position.

In one embodiment, R¹¹ is —C(O)OR¹⁴, —C(O)NHR¹⁴, —C(O)CH₂NO₂ or —C(O)CH₂SO₂R⁷

A representative example of R¹¹ is —C(O)OR¹⁴.

A representative example of R¹⁴ is hydrogen.

In one embodiment, R¹² is hydrogen, C₁₋₄alkyl optionally substituted by hydroxy, cyano, NH₂,

—NH(C₁₋₄alkyl) or —N(C₁₋₄alkyl)₂; C₂₋₄alkenyl optionally substituted by hydroxy, cyano, NH₂, —NH(C₁₋₄alkyl) or —N(C₁₋₄alkyl)₂; C₁₋₄alkoxy; C₃₋₇cycloalkyl; —NH₂; —NH(C₁₋₄alkyl);
—N(C₁₋₄alkyl)₂; (C₁₋₄alkyl)OC(O)N(C₁₋₄alkyl); or optionally substituted phenyl or benzyl;

A representative example of R¹² is C₃₋₇cycloalkyl (such as cyclopropyl), C₁₋₄alkyl (such as ethyl), C₁₋₄alkyl optionally substituted by C₁₋₄alkoxy (such as 2-methoxyethyl), or —N(C₁₋₄alkyl)₂ (such as dimethylamine).

A representative example of R¹³ is halogen, such as chlorine.

In one embodiment, R¹⁵ is hydrogen or C₁₋₄alkyl. A representative example of R¹⁵ is hydrogen or methyl.

A representative example of R¹⁶ is hydrogen.

A representative example of X is —U(CH₂)ᵥB(CH₂)ᵥD-, —U(CH₂)ᵥB—R¹⁸—, such as —U(CH₂)ᵥB—R¹⁸—.

Representative examples of U, B, D and E include the divalent radicals —N(R¹⁵)— and —O—. A representative example of R¹⁸ is C₁₋₈ alkyl (such as C₁₋₄ alkyl, more specifically ethyl or propyl).

In one embodiment U represents —O—.

In one embodiment B represents —O—.

In one embodiment D represents —N(R¹⁵)— such as —NH—.

A representative example of d is 1 to 4. In one embodiment, d is 2; in another embodiment, d is 3.

A representative example of v is 1 to 4. In one embodiment, d is 2; in another embodiment, d is 3.

In one embodiment, j is 0 or 1.

Compounds of the invention include:
4″(R)—N-(3-{2-[2-(3-Carboxy-7-chloro-1-cyclopropyl-4-oxo-1,4-dihydro-quinolin-6-ylamino)-ethoxy]-ethoxy}-propionyl}-azithromycin;
4″(S)—N-(3-{2-[2-(3-Carboxy-7-chloro-1-cyclopropyl-4-oxo-1,4-dihydro-quinolin-6-ylamino)-ethoxy]-ethoxy}-propionyl}-azithromycin;
4″(R)—N-(3-{2-[3-(3-Carboxy-1-ethyl-4-oxo-1,4-dihydro-quinolin-6-yl)-propoxy]-ethoxy}-propionyl)-azithromycin;
4″-(R)—N-(3-{2-[3-(3-Carboxy-1-dimethylamino-4-oxo-1,4-dihydro-quinolin-6-yl)-propoxy]-ethoxy}-propionyl)-azithromycin;
4″-(R)—N-(3-{2-[3-(3-Carboxy-1-(2-methoxy-ethyl-4-oxo-1,4-dihydro-quinolin-6-yl)-propoxy]-ethoxy}-propionyl)-azithromycin;
4″(S)—N-(3-{2-[3-(3-Carboxy-1-ethyl-4-oxo-1,4-dihydro-quinolin-6-yl)-propoxy]-ethoxy}-propionyl)-azithromycin;
4″(S)—N-(3-{2-[3-(3-Carboxy-1-cyclopropyl-4-oxo-1,4-dihydro-quinolin-6-yl)-propoxy]-ethoxy}-propionyl)-azithromycin; and
4″(R)—N-(3-{2-[3-(3-Carboxy-1-cyclopropyl-4-oxo-1,4-dihydro-quinolin-6-yl)-propoxy]-ethoxy}-propionyl)-azithromycin
   and pharmaceutically acceptable derivatives thereof.

Compounds according to the invention also exhibit a broad spectrum of antimicrobial activity, in particular antibacterial activity, against a wide range of clinical pathogenic microorganisms. Using a standard microtiter broth serial dilution test, compounds of the invention have been found to exhibit useful levels of activity against a wide range of clinical pathogenic microorganisms. For example, the compounds of the invention may be active against strains of Staphylococcus aureus, Streptopococcus pneumoniae, Moraxella catarrhalis, Streptococcus pyogenes, Haemophilus influenzae, Enterococcus faecalis, Chliamydia pneumoniae, Mycoplasma pneumoniae and Legionella pneumophila. The compounds of the invention may also be active against resistant strains, for example erythromycin resistant strains. For example, the compounds of the invention may be active against erythromycin resistant strains of Streptococcus pneumoniae, Streptococcus pyogenes and Staphylococcus aureus.

The compounds of the invention may therefore be used for treating a variety of diseases caused by pathogenic microorganisms, in particular bacteria, in human beings and animals. It will be appreciated that reference to treatment includes acute treatment or prophylaxis as well as the alleviation of established symptoms.

Thus, according to another aspect of the present invention we provide a compound of formula (I) or a pharmaceutically acceptable derivative thereof for use in therapy.

According to a further aspect of the invention, we provide a compound of formula (I) or a pharmaceutically acceptable derivative thereof for use in the therapy or prophylaxis of systemic or topical microbial infections in a human or animal subject.

According to a further aspect of the invention, we provide the use of a compound of formula (I) or a pharmaceutically acceptable derivative thereof in the manufacture of a medicament for use in the treatment or prophylaxis of systemic or topical microbial infections in a human or animal body.

According to a yet further aspect of the invention, we provide a method of treatment of the human or non-human animal body to combat microbial infections comprising administration to a body in need of such treatment of an effective amount of a compound of formula (I) or a pharmaceutically acceptable derivative thereof.

While it is possible that, for use in therapy, a compound of the invention may be administered as the raw chemical it is preferable to present the active ingredient as a pharmaceutical formulation eg when the agent is in admixture with a suitable pharmaceutical excipient, diluent or carrier selected with regard to the intended route of administration and standard pharmaceutical practice.

Accordingly, in one aspect, the present invention provides a pharmaceutical composition or formulation comprising at least one compound of the invention or a pharmaceutically acceptable derivative thereof in association with a pharmaceutically acceptable excipient, diluent and/or carrier. The excipient, diluent and/or carrier must be “acceptable” in the sense of being compatible with the other ingredients of the formulation and not deleterious to the recipient thereof.

In another aspect, the invention provides a pharmaceutical composition comprising, as active ingredient, at least one compound of the invention or a pharmaceutically acceptable derivative thereof in association with a pharmaceutically acceptable excipient, diluent and/or carrier for use in therapy, and in particular, in the treatment of human or animal subjects suffering from a condition susceptible to amelioration by an antimicrobial compound.

In another aspect, the invention provides a pharmaceutical composition comprising a therapeutically effective amount of the compounds of the present invention and a pharmaceutically acceptable excipient, diluent and/or carrier (including combinations thereof).

There is further provided by the present invention a process of preparing a pharmaceutical composition, which process comprises mixing at least one compound of the invention or a pharmaceutically acceptable derivative thereof, together with a pharmaceutically acceptable excipient, diluent and/or carrier.

The compounds of the invention may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable excipients, diluents and/or earners. Acceptable excipients, diluents and carriers for therapeutic use are well known in the pharmaceutical art, and are described, for example, in Remington's Pharmaceutical Sciences, Mack Publishing Co. (A. R. Gennaro edit. 1985). The choice of pharmaceutical excipient, diluent and/or carrier can be selected with regard to the intended route of administration and standard pharmaceutical practice. The pharmaceutical compositions may comprise as—or in addition to—the excipient, diluent and/or carrier any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO 91/11172, WO 94/02518 and WO 98/55148.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see International Patent Application No. WO 02/00196 (SmithKline Beecham).

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual.

There may be different composition/formulation requirements depending on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

It is to be understood that not all of the compounds need be administered by the same route. Likewise, if the composition comprises more than one active component, then those components may be administered by different routes.

The compositions of the invention include those in a form especially formulated for parenteral, oral, buccal, rectal, topical, implant, ophthalmic, nasal or genito-urinary use. For some applications, the agents of the present invention are delivered systemically (such as orally, buccally, sublingually), more preferably orally. Hence, preferably the agent is in a form that is suitable for oral delivery.

If the compound of the present invention is administered parenterally, then examples of such administration include one or more of: intravenously, intraarterially, intraperitoneally, intrathecally, intraventricularly, intraurethrally, intrasternally, intracranially, intramuscularly or subcutaneously administering the agent; and/or by using infusion techniques.

For parenteral administration, the compound is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The compounds according to the invention may be formulated for use in human or veterinary medicine by injection (e.g. by intravenous bolus injection or infusion or via intramuscular, subcutaneous or intrathecal routes) and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilising, solubilising and/or dispersing agents. Alternatively the active ingredient may be in sterile powder form for reconstitution with a suitable vehicle, e.g. sterile, pyrogen-free water, before use.

The compounds of the invention can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

The compounds of the invention may also be presented for human or veterinary use in a form suitable for oral or buccal administration, for example in the form of solutions, gels, syrups, mouth washes or suspensions, or a dry powder for constitution with water or other suitable vehicle before use, optionally with flavouring and colouring agents. Solid compositions such as tablets, capsules, lozenges, pastilles, pills, boluses, powder, pastes, granules, bullets or premix preparations may also be used. Solid and liquid compositions for oral use may be prepared according to methods well known in the art. Such compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The compounds of the invention may also be administered orally in veterinary medicine in the form of a liquid drench such as a solution, suspension or dispersion of the active ingredient together with a pharmaceutically acceptable carrier or excipient.

The compounds of the invention may also, for example, be formulated as suppositories e.g. containing conventional suppository bases for use in human or veterinary medicine or as pessaries e.g. containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g. eye ear or nose drops) or pour-ons.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water.

Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds may also be dermally or transdermally administered, for example, by use of a skin patch.

For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

As indicated, the compound of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134AT″″) or 1,1,1,2,3,3,3-heptafluoropropane (HFA 227EA), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebuliser.

The compounds of the invention may also be used in combination with other therapeutic agents. The invention thus provides, in a further aspect, a combination comprising a compound of the invention or a pharmaceutically acceptable derivative thereof together with a further therapeutic agent.

When a compound of the invention or a pharmaceutically acceptable derivative thereof is used in combination with a second therapeutic agent active against the same disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian.

The compounds of the present invention may for example be used for topical administration with other active ingredients such as corticosteroids or antifungals as appropriate. The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The compositions may contain from 0.01-99% of the active material. For topical administration, for example, the composition will generally contain from 0.01-10%, more preferably 0.01-1% of the active material.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

For oral and parenteral administration to humans, the daily dosage level of the agent may be in single or divided doses.

For systemic administration the daily dose as employed for adult human treatment it will range from 2-100 mg/kg body weight, preferably 5-60 mg/kg body weight, which may be administered in 1 to 4 daily doses, for example, depending on the route of administration and the condition of the patient. When the composition comprises dosage units, each unit will preferably contain 200 mg to 1 g of active ingredient. The duration of treatment will be dictated by the rate of response rather than by arbitrary numbers of days.

Compounds of general formula (I) and pharmaceutically acceptable derivatives thereof may be prepared by the general methods outlined hereinafter, said methods constituting a further aspect of the invention. In the following description, the groups R¹ to R¹⁸, A, B, D, E, X, Y, U, W, a, b, d, e, j, q, t, v and z have the meaning defined for the compounds of formula (I) unless otherwise stated.

The group X^{a}R^{8a} is XR⁸ as defined for formula (I) or a group convertible to XR⁸. In one preferred embodiment, X^{a} and R^{8a} are independently either X and R⁸ or the resultant of X and R⁸ after a reaction with a protecting group. In another embodiment, X^{a} and R^{8a} are independently either X and R⁸ or the resultant of X and R⁸ after reaction with an activating group. In one preferred embodiment, convertible means that the group can be formed by cleaving a protecting group from the compound. Conversion of a group X^{a}R^{8a} to a XR⁸ group typically arises if a protecting group is needed during the reactions described below. A comprehensive discussion of the ways in which such groups may be protected and methods for cleaving the resulting protected derivatives is given by for example T. W. Greene and P. G. M Wuts in Protective Groups in Organic Synthesis 2^{nd} ed., John Wiley & Son, Inc 1991 and by P. J. Kocienski in Protecting Groups, Georg Thieme Verlag 1994 which are incorporated herein by reference, Examples of suitable amino protecting groups include acyl type protecting groups (e.g. formyl, trifluoroacetyl and acetyl), aromatic urethane type protecting groups (e.g. benzyloxycarbonyl (Cbz) and substituted Cbz, and 9-fluorenylmethoxycarbonyl (Fmoc)), aliphatic urethane protecting groups (e.g. t-butyloxycarbonyl (Boc), isopropyloxycarbonyl and cyclohexyloxycarbonyl) and alkyl type protecting groups (e.g. benzyl, trityl and chlorotrityl). Examples of suitable oxygen protecting groups may include for example alkyl silyl groups, such as trimethylsilyl or tert-butyldimethylsilyl; alkyl ethers such as tetrahydropyranyl or tert-butyl; or esters such as acetate. Hydroxy groups may be protected by reaction of for example acetic anhydride, benzoic anhydride or a trialkylsilyl chloride in an aprotic solvent. Examples of aprotic solvents are dichloromethane, N,N-dimethylformamide, dimethylsulfoxide, tetrahydrofuran and the like.

Compounds of formula (I) wherein d is an integer from 1 to 5 may be prepared by reaction of a 4″ amine compound of formula (II)

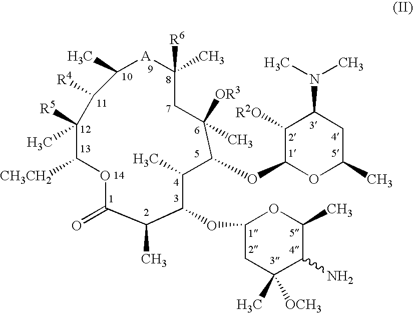

with a carboxylic acid compound of formula (III),

HOC(O)(CH₂)_{d}X^{a}R^{8a} (III)

or a suitable activated and protected derivative thereof, followed where necessary by subsequent conversion of the X^{a}R^{8a} group to XR⁸.

Suitable activated derivatives of the carboxyl group include the corresponding acyl halide, mixed anhydride or activated ester such as a thioester. The reaction is preferably carried out in a suitable aprotic solvent such as a halohydrocarbon (e.g. dichloromethane) or N,N-dimethylformamide optionally in the presence of a tertiary organic base such as dimethylaminopyridine or triethylamine or in the presence of inorganic base (eg sodium hydroxide) and at a temperature within the range of 0° to 120° C. The compounds of formula (II) and (III) may also be reacted in the presence of a carbodiimide such as dicyclohexylcarbodiimide (DCC) or 1-(3-dimethylaminopropyl)-3-ethylcarbodiimide (EDC).

Compounds of formula (I) wherein d is 0 and U is —NH— may be prepared by reaction of the 4″ amine of formula (II) with a suitable activated derivative such as the isocyanate OCNX^{a}R^{8a}.

Compounds of formula (I) wherein d is 0 and U is —N(R¹⁵)— may be prepared by reaction of the 4″ amine of formula (II) with a suitable activated derivative such as the carbamoyl chloride ClC(O)N(R¹⁵)X^{a}R^{8a}.

In a further embodiment of the invention, compounds of formula (I) wherein d is an integer from 1 to 5 and U is a group selected from —N(R¹⁵)—, —O— and —S—, may be prepared by reaction of compounds of formula (IV)

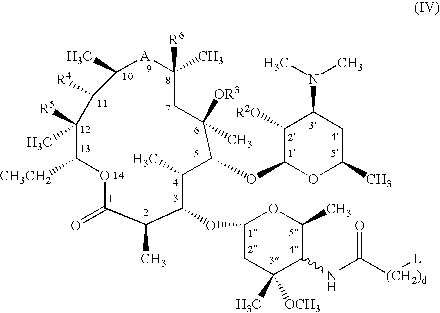

wherein d is an integer from 1 to 5 and L is a suitable leaving group, with a compound of formula (V)

X^{a}R^{8a} (V)

in which U is a group selected from —N(R¹⁵)—, —O— and —S—.

The reaction is preferably carried out in a solvent such as a halohydrocarbon (e.g. dichloromethane), an ether (e.g. tetrahydrofuran or dimethoxyethane), acetonitrile or ethyl acetate and the like, dimethylsulfoxide, N,N-dimethylformamide or 1-methyl-pyrrolidone and in the presence of a base, followed, if desired, by conversion of the X^{a}R^{8a} group to XR⁸. Examples of the bases which may be used include organic bases such as diisopropylethylamine, triethylamine and 1,8-diazabicyclo[5.4.0]undec-7-ene, and inorganic bases such as potassium hydroxide, cesium hydroxide, tetraalkylammonium hydroxide, sodium hydride, potassium hydride and the like. Suitable leaving groups for this reaction include halide (e.g. chloride, bromide or iodide) or a sulfonyloxy group (e.g. tosyloxy or methanesulfonyloxy).

Compounds of formula (IV) may be prepared by reaction of a compound of formula (II) with a carboxylic acid of formula (VI)

HOC(O)(CH₂)_{d}L (VI),

wherein L is a suitable leaving group as above defined, or a suitable activated thereof.

Suitable activated derivatives of the carboxyl group are those defined above for carboxylic acid (III). The reaction is carried out using the conditions described above for the reaction of a compound of formula (II) with carboxylic acid (III).

In one preferred embodiment, the compound of formula (I) is prepared by reacting a compound of formula (II) with a suitable derivative of formula (III), an isocyanate, or a carbamoyl chloride or reacting a compound of formula (IV) with a compound of the formula (V), wherein both of X^{a} and R^{8a} are X and R⁸ as defined for formula (I) or they are independently protecting groups that may be cleaved to form the variable as defined in formula (I).

Compounds of formula (I) may be converted into other compounds of formula (I). Thus compounds of formula (I) wherein U, B, D or E is —S(O)_{z}— and z is 1 or 2 may be prepared by oxidation of the corresponding compound of formula (I) wherein z is 0. The oxidation is preferably carried out using a peracid, e.g. peroxybenzoic acid, followed by treatment with a phosphine, such as triphenylphosphine. The reaction is suitably carried out in an organic solvent such as methylene chloride.

Compounds of formula (I) wherein U, B, D or E is —N(R¹⁵)— and R¹⁵ is C₁₋₄alkyl can be prepared from compounds of formula (I), wherein R¹⁵ is hydrogen by reductive alkylation.

Compounds of formula (II) wherein A is —C(O)NH— or —NHC(O)—, R⁴ or R⁵ are hydroxy, R³ is hydrogen and R⁶ is hydrogen are known compounds or they may be prepared by analogous methods to those known in the art. Thus they can be prepared according to the procedures described in EP 507595 and EP 503932.

Compounds of formula (II), wherein A is —C(O)NH— or —NHC(O)—, R⁴ or R⁵ are hydroxy and R³ is C₁₋₄alkyl or C₃₋₆alkenyl optionally substituted by 9 to 10 membered fused) bicyclic heteroaryl and R⁶ is hydrogen are known compounds or they may be prepared by analogous methods to those known in the art. Thus they can be prepared according to the procedures described in WO 9951616 and WO 0063223.

Compounds of formula (II), wherein A is —C(O)NH—, R⁴ and R⁵ taken together with the intervening atoms form a cyclic group having the following structure:

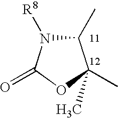

R³ is C₁₋₄alkyl, or C₃₋₆alkenyl optionally substituted by 9 to 10 membered fused bicyclic heteroaryl and R⁶ is hydrogen are known compounds or they may be prepared by analogous methods to those known in the art. Thus they can be prepared according to the procedures described in U.S. Pat. No. 6,262,030.

Compounds of formula (II), wherein A is —C(O)NH—, —NHC(O)—, —N(CH₃)—CH₂— or —CH₂—N(CH₃)—, R⁴ or R⁵ are hydroxy or R⁴ and R⁵ taken together with the intervening atoms form a cyclic group having the following structure:

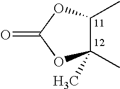

and R⁶ is hydrogen are known compounds or they may be prepared by analogous methods to those known in the art. Thus they can be prepared according to the procedures described in EP 508699 and J. Chem. Res. Synop. (1988 pages 152-153), U.S. Pat. No. 6,262,030.

Compounds of formula (II), wherein A is —C(O)NH—, R⁴ and R⁵ taken together with the intervening atoms form a cyclic group having the following structure:

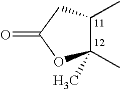

R⁶ is hydrogen and R³ is C₁₋₄ alkyl may be prepared by decarboxylation of a compound of formula (VII):

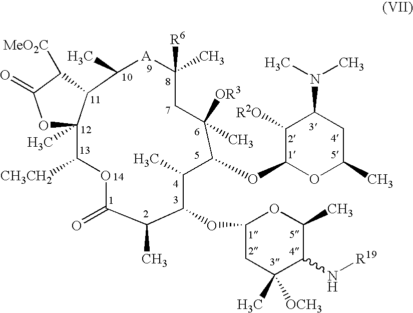

wherein R¹⁹ is amino protecting group. The decarboxylation is followed, if required, by the removal of the protecting group R¹⁹.

The decarboxylation may be carried out in the presence of a lithium salt such as lithium chloride, preferably in an organic solvent such as dimethylsulfoxide.

Compounds of formula (II), wherein A is —C(O)NH—, R⁴ and R⁵ taken together with the intervening atoms form a cyclic group having the following structure:

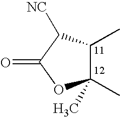

and R₃ is C₁₋₄ alkyl may be prepared according to the procedures described in WO 02/50091 and WO 02/50092.

Compounds of formula (III) wherein X is —U(CH₂)ᵥB(CH₂)ᵥD-, —U(CH₂)ᵥB—R¹⁸—, —U(CH₂)ᵥB(CH₂)ᵥD(CH₂)ᵥE-, —U(CH₂)ᵥB(CH₂)ᵥD-R⁸—, in which U, B, D and E are —N(R¹⁵)—, —O—, —S—, or X is a group selected from:

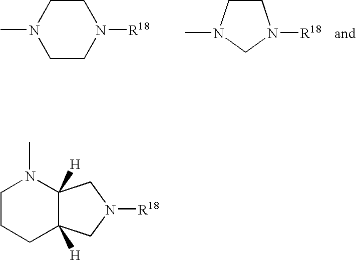

may be prepared by reaction of a compound of formula (V), as defined above, with a compound of formula (VIII):

R²⁰OC(O)CH═CH₂ (VIII)

wherein R²⁰ is carboxyl protecting group, followed by removal of R²⁰.

Suitable R²⁰ carboxyl protecting group include t-butyl, allyl or benzyl.

Compounds of formula (III) may also be prepared by reaction of a compound of formula (V), as defined above, with acrylonitrile followed by hydrolysis of the nitrile to the acid.

Compounds of formula (V) wherein X is:
—U(CH₂)ᵥB(CH₂)ᵥD- in which D is —N(R¹⁵)—, —O— or —S—; or
—U(CH₂)ᵥB(CH₂)ᵥD(CH₂)E- in which E is —N(R¹⁵)—, —O— or —S—; or

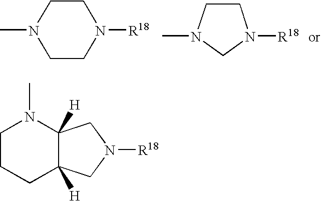

may be prepared by reaction of a compound of formula (IX)

R^{8a}L (IX)

wherein L is a suitable leaving group such as chlorine, fluorine, iodine or bromine, with a compound of formula (X)

—U(CH₂)ᵥB(CH₂)ᵥD- (X)

wherein D is —N(R¹⁵)—, —O— or —S—
or with compound of formula (XI)

—U(CH₂)ᵥB(CH₂)ᵥD(CH₂)ᵥE- (XI)

wherein E is —N(R¹⁵)—, —O— or —S—
or with piperazine
or with imidazolidin
or with 1H-octahydro-pyrrolo[3,4-b]pyridine.

Compound of formula (IX), as defined above, wherein L is a suitable leaving group such as chlorine, fluorine or bromine, and R¹⁶ and R¹² are linked to form the bivalent radical —O(CH₂)₂—, —(CH₂)ₜ—; —NR⁷(CH₂)ₐ—, —OCH₂NR⁷—, —SCH₂NR⁷—, —CH₂NR⁷CH₂—, —CH₂OCH₂—, —CH₂SCH₂— or —(CH₂)ₐNR⁷— are known compounds or they may be prepared by analogous methods to those known in the art. Thus, they can be prepared according to the procedures described in U.S. Pat. No. 6,624,159.

Compound of formula (IX), as defined above, wherein L is a suitable leaving group such as chlorine, fluorine or bromine, and R¹⁷ and R¹² are linked to form the bivalent radical selected from the group —(CH₂)_{b}S—, —(CH₂)_{b}NR⁷— or —(CH₂)_{b}O— are known compounds or they may be prepared by analogous methods to those known in the art. Thus, they can be prepared according to the procedures described in Arch. Pharm. Pharm. Med. Chem. 330, 63 (1997).

In order that the invention may be more fully understood, the following examples are given by way of illustration only. In the structures shown herein, the pendent oxygens shown as —O are meant to include hydroxyl substitutions (i.e., —OH).

The 4″-configuration (S) and (R) of amino intermediates 20A and 20B was arbitrarily assigned based on NMR data. Consequently all 4″-amido compound examples have arbitrarily assigned configuration (S) and (R) as well depending on starting 4″-amino intermediate.

All references contained in this application are herein incorporated by reference in their entireties.

The following abbreviations are used in the text: DBU for 1,8-diazabicyclo[5.4.0]undec-7-ene, DCC for dicyclohexylcarbodiimide, DCM for dichloromethane, DMAP for 4-dimethylaminopyridine, DMF for N,N-dimethylformamide, DMSO for dimethyl sulfoxide, EtOAc for ethyl acetate, EtOH for ethanol, KO^{t}Bu for potassium tert-butoxide, MeOH for methanol, i-PrOH for isopropanol, and MIBC for methyl isobutyl ketone, HOBT for 1-hydroxy benzotriazole hydrate, EDC for 1-(3-dimethylaminopropyl)-3-ethylcarbodiimide, DIPE for diisopropylether.

### EXAMPLES

4″-(S) and 4″-(R)-Amino-azithromycin may be prepared by the procedure described in EP 508 699. 4″-Keto-azithromycin may be prepared using the Pfitzner-Moffat-procedure (J. Am. Chem. Soc., 87, 5670-5678, 1965) at room temperature for 4 hours and deprotecting in MeOH. 1-Cyclopropyl-6-iodo-4-oxo-1,4-dihydro-quinoline-3-carboxylic acid ethyl ester, 7-chloro-1-isopropyl-6-fluoro-4-oxo-1,4-dihydro-quinoline-3-carboxylic acid, 7-chloro-1-tert-butyl-6-fluoro-4-oxo-1,4-dihydro-quinoline-3-carboxylic acid, 1-dimethylamino-6-iodo-4-oxo-1,4-dihydro-quinoline-3-carboxylic acid, 1-(2-methoxyethyl)-6-iodo-4-oxo-1,4-dihydro-quinoline-3-carboxylic acid may be prepared according to the procedure described in J. Med. Chem. 1995, 38, 973. 1-Ethyl-6-iodo-4-oxo-1,4-dihydro-quinoline-3-carboxylic acid ethyl ester my be prepared by procedure described in Aust. J. Chem., 1973, 26, 907.

### Intermediate 1:

### 7-{2-[2-(2-Carboxy-ethoxy)-ethoxy]-ethylamino}-1,2,3,6-tetrahydro-6-oxo-[1,3]-oxazino-[3,2a]-quinoline-5-carboxylic acid

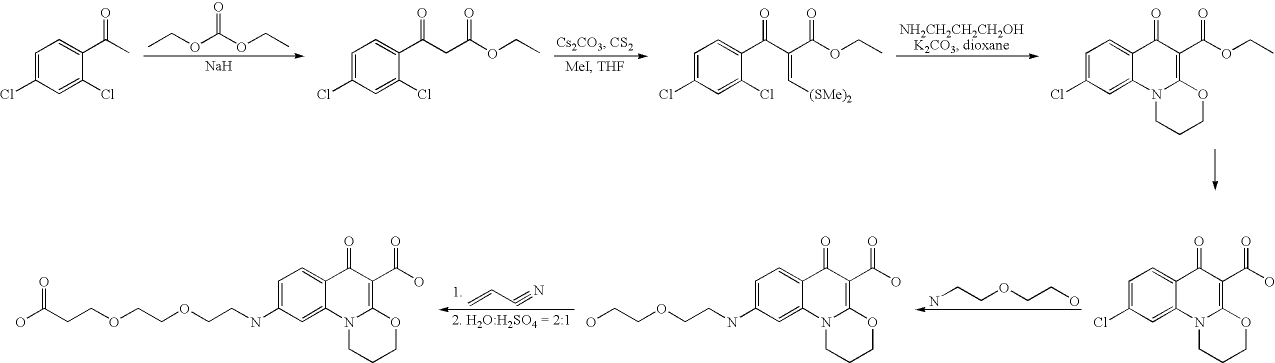

### a) 3-(2,4-Dichlorophenyl)-3-oxo-propionic acid ethyl ester

Synthesis of Intermediate 1a was done by standard procedure starting from 2,4-dichloroacetophenone, diethylcarbonate (25 eq) and NaH (2 eq) at 80° C. for 60 minutes.

MS (ES+) m/z: [MH]⁺=262

### b) 2-[Bis(methylthio)methylene]-3-(2,4-dichlorophenyl)-3-oxo-propionic acid ethyl ester

To a mixture of Intermediate 1a (15.7 g) and Cs₂CO₃ (2.5 eq) in THF (230 mL) was added CS₂ (4.6 eq) with stirring at −10° C. After 5 minutes CH₃I (2.5 eq) was added in one portion and reaction was stirred at room temperature overnight. The reaction was diluted with ether (50 mL) and filtered. Filtrate was concentrated in vacuo.

MS (ES+) m/z: [MH]⁺=366

### c) 7-Chloro-1,2,3,6-tetrahydro-6-oxo-[1,3]oxazino[3,2a]quinoline-5-carboxylic acid ethyl ester

A mixture of Intermediate 1b (18.08 g), 3-amino-1-propanole (1.2 eq) and K₂CO₃ (2.4 eq) in dioxane (500 mL) was stirred at room temperature for 1 hour and refluxed overnight. The reaction mixture was filtrated and filtrate was concentrated to dryness under reduced pressure. The crude product was precipitated from MeOH affording the title compound (2.6 g).

MS (ES+) m/z: [MH]⁺=308

### d) 7-Chloro-1,2,3,6-tetrahydro-6-oxo-[1,3]oxazino[3,2a]quinoline-5-carboxylic acid

To a solution of Intermediate 1c (1.4 g) in THF (15 mL) solution of NaOH (4.6 eq) in water (15 mL) was added and the reaction mixture was stirred at 80° C. overnight. THF was evaporated, HCl (0.6 M) was added to reach pH value about 4 and extracted with 3×10 mL of DCM. The organic layers were washed with brine, dried over Na₂SO₄, filtered and DCM was evaporated under reduced pressure affording the title compound (1.16 g).

MS (ES+) m/z: [MH]⁺=280

### e) 7-[2-(2-Hydroxy-ethoxy)-ethylamino]-1,2,3,6-tetrahydro-6-oxo-[1,3]oxazino[3,2a]-quinoline-5-carboxylic acid

Intermediate 1d (1 g) was diluted in 5 mL of methyl-pyrrolidone, 1.8 mL (5 eq) of 2-(2-aminoethoxy)ethanol was added and stirred at 110° C. for 24 hours. To the reaction mixture was added EtOAc, the pH was adjusted to 6 and extracted with 3×15 mL of H₂O. The organic layers were washed with brine, dried over Na₂SO₄, filtered and EtOAc was evaporated under reduced pressure affording the title compound (600 mg).

MS (ES+) m/z: [MH]⁺=349

### f) 7-{2-[2-(2-Carboxy-ethoxy)-ethoxy]-ethylamino}-1,2,3,6-tetrahydro-6-oxo-[1,3]-oxazino-[3,2a]-quinoline-5-carboxylic acid

Intermediate 1e (600 mg) was diluted in 7.4 mL of C₃H₃N, 0.515 mL of DBU was added and the mixture was stirred at 80° C. for 24 hours. C₃H₃N was evaporated under reduced pressure, residue dissolved in EtOAc, the pH was adjusted to 3 and extracted with 3×15 mL of H₂O. EtOAc was evaporated under reduced pressure affording 650 mg of cyano derivative. The cyano derivative was dissolved in 40 mL of H₂O/H₂SO₄ (2:1) and stirred for 24 h at 75° C. affording the title compound.

MS (ES+) m/z: [MH]⁺=421

### Intermediate 2

### 1Cyclopropyl-6-fluoro-7-chloro-4-oxo-1,4-dihydro-quinoline-3-(2-nitroacetyl)

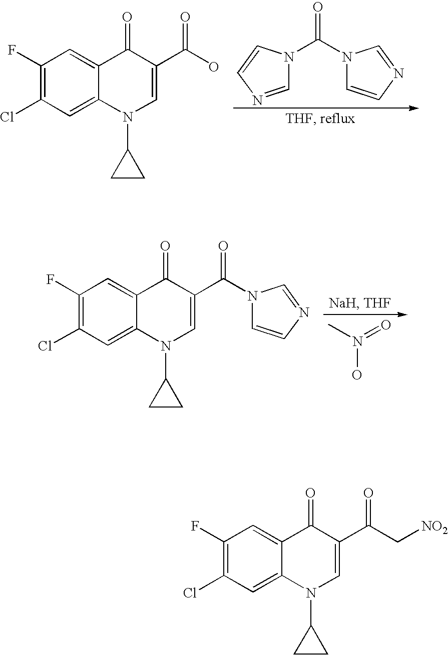

A mixture of 7-chloro-1-cyclopropyl-6-fluoro-4-oxo-1,4-dihydro-quinoline-3-carboxylic acid (1 g, 3.55 mmol) and of 1,1-carbonyldiimidazole (2.88 g, 17.75 mmol) in 15 ml CCl₃ was heated to reflux over the night. The mixture was cooled and the solvent was removed under reduced pressure. To the residue a small amount of diethyl ether was added and the resulting solid was collected by filtration and washed with diethyl ether to give an imidazolide intermediate in a quantitative yield.

To the mixture of NaH (0.26 g, 0.0108 mol, 60% disperse oil) and of nitromethane (0.58 ml 0.0108 mol) in 20 ml of anhydrous THF a solution of imidazolide intermediate (0.9 g, 0.289 mmol) in 20 ml of anhydrous THF was added dropwise and heated to reflux for 18 h. The mixture was cooled and 20 ml of H₂O was slowly added and neutralized by HCl, and then extracted with CH₂Cl₂. The organic layer was washed with H₂O and brine, dried by anhydrous Na₂SO₄ and evaporated. The product was precipitated and filtrated off yielding 0.4 g of title compound. (90.6% pure compound according to LC-MS).

MS (ES+) m/z: [MH]+=325.1.

### Intermediate 3

### 1-Cyclopropyl-6,7-difluoro-8-methoxy-4-oxo-1,4-dihydro-quinoline-3-(2-nitroacetyl)

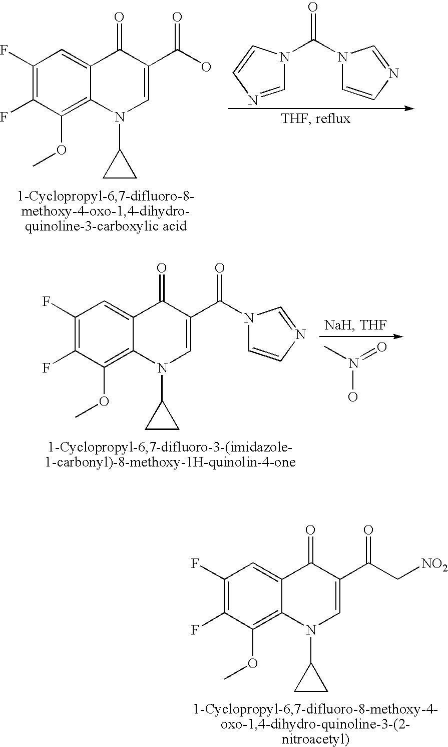

A mixture of 1-cyclopropyl-6,7-difluoro-8-methoxy-4-oxo-1,4-dihydro-quinoline-3-carboxylic acid (1 g, 3.38 mmol) and 1,1-carbonyldiimidazole (2.19 g, 13.54 mmol) in 15 ml CCl₃ was heated to reflux over the night. The mixture was cooled and the solvent was removed under reduced pressure. To the residue a small amount of diethyl ether was added and the resulting solid was collected by filtration and washed with diethyl ether to give an imidazolide intermediate in a quantitative yield.

To the mixture of NaH (0.28 g, 0.0116 mmol, 60% disperse oil) and nitromethane (0.62 ml, 0.01158 mol) in 20 ml of anhydrous THF a solution of imidazolide intermediate (1 g, 2.89 mmol) in 20 ml of anhydrous THF was added dropwise and heated to reflux for 18 h. The mixture was cooled and 20 ml of H₂O was slowly added and neutralized by HCl, and then extracted with CH₂Cl₂. The organic layer was washed with H₂O and brine, dried by anhydrous Na₂SO₄ and evaporated. The product was precipitated and filtrated off yielding 0.56 g of title product. (93.46% pure compound according to LC-MS).

MS (ES+) m/z: [MH]⁺=339.1.

### Intermediate 4

### 7-[2-(2-Cyano-ethoxy)-ethylamino]-1-cyclopropyl-6-fluoro-8-methoxy-4-oxo-1,4-dihydro-quinoline-3-(2-nitroacetyl)

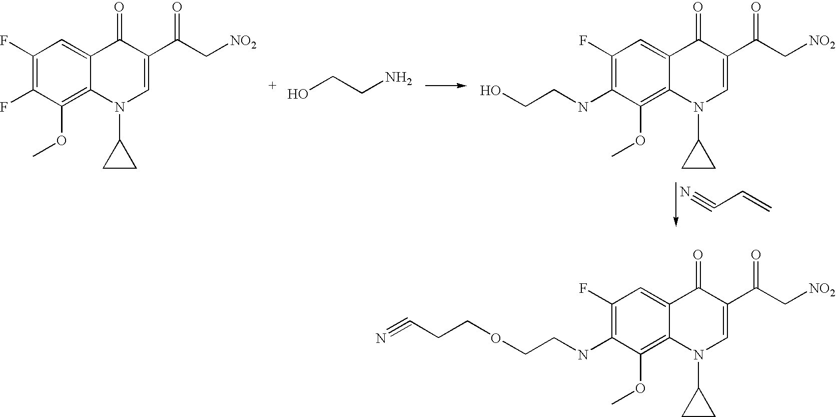

To a solution of Intermediate 3 (250 mg) in DMSO (15 ml) ethanolamine (0.425 ml) was added and the reaction mixture Was stirred at 90° C. for 1.5 hours. pH Value of mixture was adjusted to 4.5 and product was precipitated. After filtration, 190 mg of 1-cyclopropyl-6-fluoro-7-(2-hydroxy-ethylamino)-8-methoxy-4-oxo-1,4-dihydro-quinoline-3-(2-nitroacetyl) was obtained. A solution of 1-cyclopropyl-6-fluoro-7-(2-hydroxy-ethylamino)-8-methoxy-4-oxo-1,4-dihydro-quinoline-3-(2-nitroacetyl) (180 mg) in acrylonitrile and DBU was stirred at 80° C. under N₂ for 5 hours. CH₃CN was evaporated under reduced pressure yielding oily title product.

### Intermediate 5

### 6-[3-piperazin-1-yl)-propyl]-1-ethyl-4-oxo-1,4-dihydro-quinoline-3-carboxylic acid ethyl ester

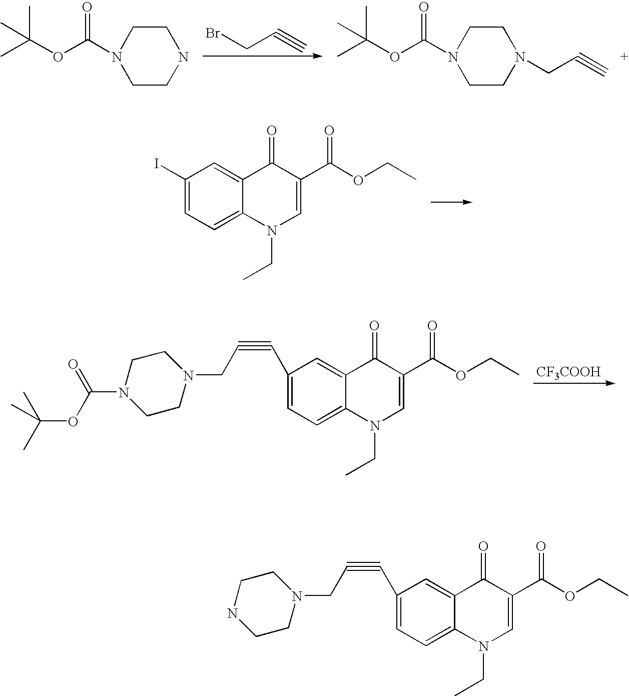

### a) 4-Prop-2-ynyl-piperazine-1-carboxylic acid tert-butyl ester

To the degassed solution of piperazine-1-carboxylic acid tert-butyl ester (1.0 g, 5.37 mmol) in acetonitrile (10 ml) were added Na₂CO₃ (1.708 g, 16.11 mmol) and mixture was stirred for min. The suspension was heated to 50° C. and 3-bromo-propyne (0.9 mL, 8.055 mmol) was added. The solvent was evaporated and the residue was extracted with Et-Ac and water (2×50 mL). Organic layer was washed with NaCl and NaHCO₃ (2×50 ml). The organic layer was dried over K₂CO₃ and evaporated in vacuum yielding (0.70 g) oil title intermediate.

MS (ES+) m/z: [MH]⁺=225.1.

### b) 6-[3-(4-tert-Butoxycarbinyl-piperazin-1-yl)-prop-1-ynyl]-1-ethyl-4-oxo-1,4-dihydro-quinoline-3-carboxylic acid ethyl ester

1-Ethyl-6-iodo-4-oxo-1,4-dihydro-quinoline-3-carboxylic acid ethyl ester (0.7 g, 3.125 mmol), copper (I) iodide (42.47 mg, 0.223 mmol) and triethylamine (10.809 mL, 78.05 mmol) were suspended in dry acetonitrile (20 ml). The suspension was heated to 50° C. and N₂ bubbled through. After 20 min, dichlorobis (triphenylphosphine) palladium (II) (46.96 mg, 0.0669 mmol) and Intermediate 5a (0.7 g 3.125 mmol) were added and dark red suspension was heated at 50° C. for 3 hours. The solvent was evaporated and the residue was extracted with EtOAc and water (2×50 mL). Organic layer was washed with NaCl and NaHCO₃ (2×50 mL), dried over K₂CO₃ and evaporated in vacuum yielding (1.24 g) oil red title product.

MS (ES+) m/z: [MH]⁺=468.3.

### c) 6-[3-piperazin-1-yl)-propyl]-1-ethyl-4-oxo-1,4-dihydro-quinoline-3-carboxylic acid ethyl ester

To the solution of Intermediate 5b (1.2 g, 2.57 mmol) in DCM (1.2 mL) was added CF₃COOH (1.2 mL) and mixture was stirred at room temperature for 48 h. To the reaction mixture was added water (pH=1.2) and layers were separated (H=9.6). The organic layer was dried over K₂CO₃ and evaporated in vacuum yielding (1.7 g) oil red title product.

MS (ES+) m/z: [MH]⁺=368.3.

### Intermediate 6

### 1-Cyclopropyl-6-fluoro-7-[2-(2-hydroxy-ethoxy)-ethylamino]-4-oxo-1,4-dihydro-quinoline-3-carboxylic acid (A) and

### 7-Chloro-1-cyclopropyl-6-[2-(2-hydroxy-ethoxy)-ethyl amino]-4-oxo-1,4-dihydro-quinoline-3-carboxylic acid (B)

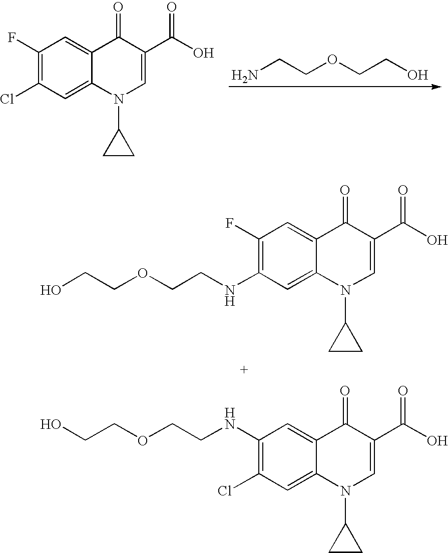

To a mixture of 7-chloro-1-cyclopropyl-6-fluoro-4-oxo-1,4-dihydro-quinoline-3-carboxylic acid (10 g, 0.035 mol) in 1-methyl-2-pirolidone (70 mL) 2-(2-amino-ethoxy)-ethanol (18 mL, 0.18 mol, 5 eq.) was added, the reaction mixture was stirred at 110° C. for 24 hours. Then was diluted with water (200 mL) and CH₂Cl₂ (60 mL) and the pH was adjusted to 10. The aqueous layer was extracted with CH₂Cl₂ (5×50 mL) and then the pH was adjusted to 6.7. After 10 minutes first product precipitated. Filtrated off yielding 2.7 g of crude 7-chloro-1-cyclopropyl-6-[2-(2-hydroxy-ethoxy)-ethylamino]-4-oxo-1,4-dihydro-quinoline-3-carboxylic acid. (according to LC-MS 100% pure Intermediate 6B) Over night second product precipitated. Filtrated off yielding 7.7 g of yellow product (according to LC-MS a mixture of Intermediate 6A and Intermediate 6B in a 1:1 ratio).

### Intermediate 7

### 6-{2-[2-(2-carboxy-ethoxy)ethoxy]ethylamino}-1-cyclopropyl-7-chloro-4-oxo-1,4-dihydro-quinoline-3-carboxylic acid

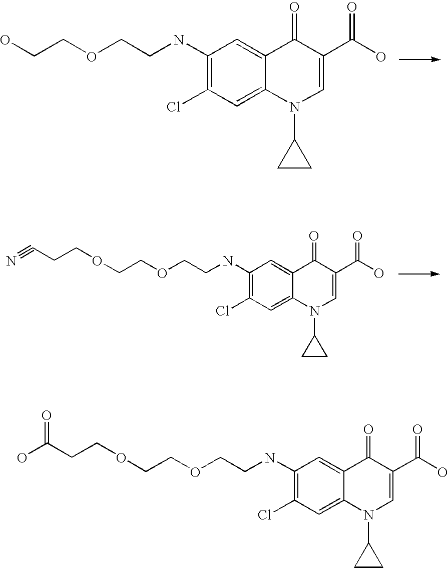

Intermediate 6B (2 g, 5.45 mmol) was diluted in 25 mL of acrylonitrile, DBU (2.0 mL) was added and stirred at 80° C. for 24 hours. Acrylonitrile was evaporated under reduced pressure, residue was dissolved in DCM, the pH was adjusted to pH 3 and extracted with 3×20 mL H₂O. The organic layers were washed with brine, dried over Na₂SO₄, filtered and DCM was evaporated under reduced pressure affording 1.9 g of 6-{2-[2-(2-cyano-ethoxy)ethoxy]ethylamino}-1-cyclopropyl-7-chloro-4-oxo-1,4-dihydro-quinoline-3-carboxylic acid. This product was dissolved in 40 mL of mixture H₂O/H₂SO₄ (1:1) and stirred for 24 hours at 75° C. The obtained precipitate was filtered and dried under reduced pressure for 1 h affording 1.7 g of title product.

### Intermediate 8

### 1-Cyclopropyl-6-fluoro-7-chloro-4-oxo-1,4-dihydro-3-[(2-methanesulfonyl)acetyl]-quinoline

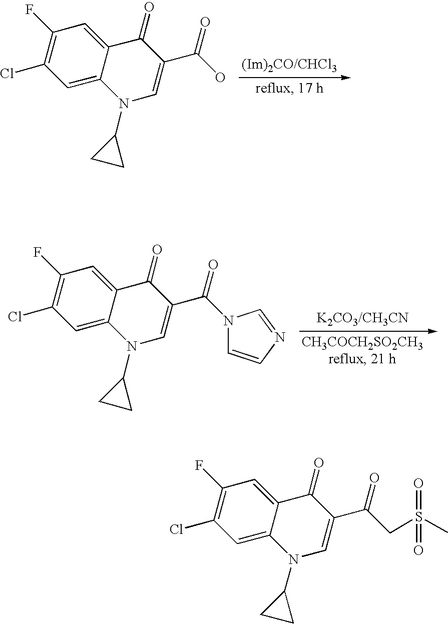

A mixture of 1-cyclopropyl-6-fluoro-7-chloro-4-oxo-1,4-dihydro-quinoline-3-carboxylic acid (2 g, 0.0071 mol) and 1,1′-carbonyldiimidazole (5.76 g, 0.035 mol) in 15 mL CHCl₃ was heated to reflux for 17 hours. The solvent was removed by reduced pressure. To the residue, ether was added and then stirred at room temperature for 30 min. The solid was filtered and dried affording 1.64 g of 3-imidazolide derivative. Imidazolide derivative (1 g, 0.003 mol) was dissolved in acetonitrile (40 ml), then methanesulphonylacetone (2 g, 0.015 mol) and K₂CO₃ were added and the mixture was heated to reflux for 21 hours. The solvent was removed under reduced pressure and 120 mL of H₂O was added. The solution was acidified by 2N HCl (pH˜3) and extracted with EtOAc. The organic layer was dried and concentrated to give a crude solid product. The crude product was purified by column chromatography (DCM-EtOH—NH₄OH=90:9:1.5) to give pure product 1-cyclopropyl-6-fluoro-7-chloro-4-oxo-1,4-dihydro-3-[(2-methanesulfonyl)acetyl]-quinoline.

MS (ES+) m/z: [MH]⁺=358.1

¹H NMR (500 MHz, DMSO) [symbol] 8.58, 8.37, 8.13, 5.22, 3.78, 3.13, 1.31 and 1.16.

### Intermediate 9

### 4″-(S)-amino-azithromycin 11,12-cyclic carbonate

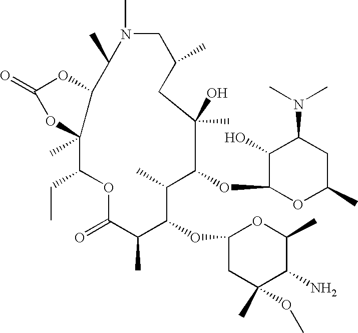

4″(S)-Amino-azithromycin (0.1 g, 0.13 mmole) was dissolved in benzene (4 mL) and then ethylene carbonate (0.09 g) and K₂CO₃ (0.11 g) were added into the reaction mixture. The reaction mixture was heating at 80° C. over night. After filtration filtrate was rinsed 2×H₂O and evaporated giving 0.097 mg of the title product.

MS (ES+) m/z: [MH]⁺=774.4.

### Intermediate 10

### 4″-(R)-amino-azithromycin 11,12-cyclic carbonate

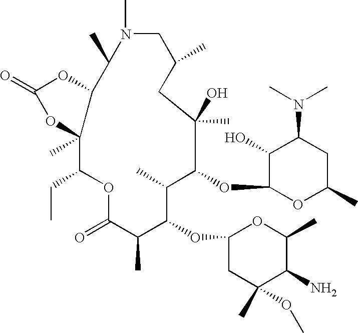

Starting from 4″(R)-amino isomer (0.080 g) cyclic carbonate was prepared using the procedure of Intermediate 9.

MS (ES+) m/z: [MH]⁺=774.4.

### Intermediate 11

### 6-[3-(2-Amino-ethoxy)-prop-1-ynyl]-1-ethyl-4-oxo-1,4-dihydro-quinoline-3-carboxylic acid trifluoroacetate salt

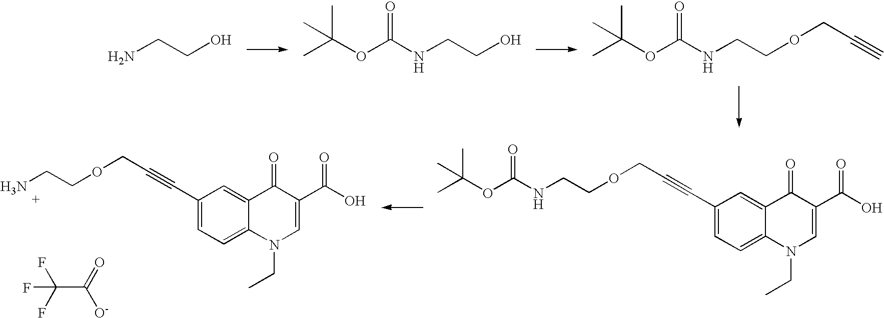

### a) (2-Hydroxy-ethyl)-carbamic acid tert-butyl ester

To a stirring solution of ethanolamine (1.96 mL, 32.7 mmol) in dioxane (40 mL) and water (20 mL) saturated solution of NaHCO₃ (20 mL) was added. The solution was cooled in ice bath and di-t-butyl dicarbonate (8.0 g) was added portionwise. After 1 hour, TLC showed no starting material. EtOAc (50 mL) and water (20 mL) were added; the organic layer was separated and evaporated yielding 4.20 g of the oily title compound.

### b) (2-Prop-2-ynyloxy-ethyl)-carbamic acid tert-butyl ester

To a stirring solution of Intermediate 11a (1.16 g) in THF (30 mL) at room temperature t-butylammonium iodide (0.15 g), sodium iodide (0.15 g) and propargyl bromide (80% in toluene, 1.20 mL) were added. KOH (0.40 g) was added portionwise during 30 minutes and the suspension was stirred at room temperature for 24 hours. The solvent was evaporated, EtOAc (30 mL) and water (30 mL) were added, organic layer was washed with 10% Na₂S₂O₅ solution and evaporated yielding 1.21 g of the title compound.

### c) 6-[3-(2-tert-Butoxycarbonylamino-ethoxy)-prop-1-ynyl]-1-ethyl-4-oxo-1,4-dihydro-quinoline-3-carboxylic acid

CuI (55 mg) and triethylamine (14.06 mL) were added into a solution of 1-ethyl-6-iodo-4-oxo-1,4-dihydro-quinoline-3-carboxylic acid (1.0 g) in MeCN (20 mL). The mixture has been stirring at room temperature for 20 minutes. Pd(PPh₃)₂Cl₂ (61 mg) and Intermediate 11b (0.70 g) were added and the mixture has been stirring at 50° C. for 4 hours. The solvents were evaporated, EtOAc (30 mL) and water (30 mL) were added, organic layer was washed with water (30 mL) and brine (30 ml) and evaporated yielding 1.0 g of the title compound.

MS (ES+) m/z: [MH]⁺=415.24

### d) 6-[3-(2-Amino-ethoxy)-prop-6-ynyl]-1-ethyl-4-oxo-1,4-dihydro-quinoline-3-carboxylic acid trifluoroacetate salt

Trifluoroacetic acid (0.386 mL) was added into solution of Intermediate 11c (0.42 g) in MeCN (5 mL) at room temperature. The solution has been stirring at room temperature for 48 hours and evaporated yielding 0.80 g of the title compound.

### Intermediate 12

### 9-(2-hydroxy-ethylamino)-1-oxo-6,7-dihydro-1H,5H-pyrido[3,2,1-ij]quinoline-2-carboxylic acid

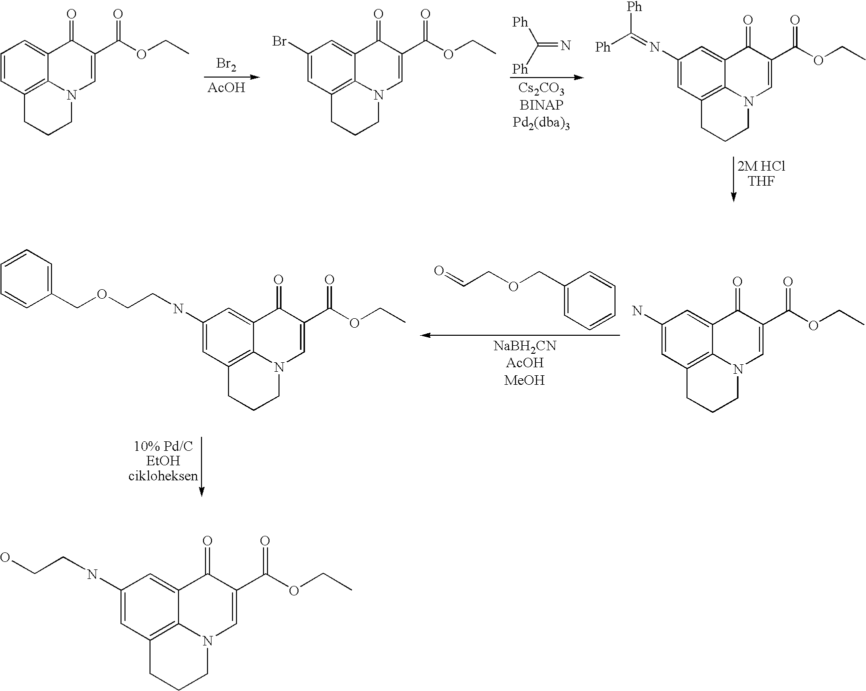

### a) 9-Bromo-1-oxo-6,7-dihydro-1H,5H-pyrido[3,2,1-ij]quinoline-2-carboxylic acid ethyl ester

To the solution of 1-Oxo-6,7-dihydro-1H,5H-pyrido[3,2,1-ij]quinoline-2-carboxylic acid ethyl ester (7.5 g, 29 mmol) in glacial acetic acid (120 mL) was added bromine (1.6 ml, 32 mmol).

The mixture was stirred over night at room temperature, and new portion of bromine (1.6 mL, 32 mmol) was added. After 24 h, reaction mixture was diluted with 100 mL of H₂O and pH was adjusted to 2.9. Precipitate was filtered and dried. The crude product was precipitated from CH₂Cl₂/Diisopropylether and dried in vacuum drier yielding 13.07 g of the crude title product.

MS (ES+) m/z: [MH]⁺=338.0.

### b) 9-(Benzhydrylidene-amino)-1-oxo-6,7-dihydro-1H,5H-pyrido[3,2,1-ij]quinoline-2-carboxylic acid ethyl ester

Tris(dibenzylideneacetone)dipalladium chloroform complex (50 mg, 0.05 mmol), rac-2,2′-bis(diphenylphosphino)-1,1′-binaphthyl (100 mg, 0.16 mmol), Intermediate 12a (3 g, 8.9 mmol) and benzophenone imine (1.2 ml) were diluted in THF (45 ml). The air of atmosphere was replaced with N₂, and Cs₂CO₃ (2.5 g) was added. The mixture was stirred under reflux. Another two portions of Tris(dibenzylideneacetone)dipalladium chloroform complex (50 mg, 0.05 mmol), rac-2,2′-bis(diphenylphosphino)-1,1′-binaphthyl (100 mg, 0.16 mmol), benzophenone imine (1.2 ml) and Cs₂CO₃ (2.5 g) was added every 2.5 h. The mixture was stirred under reflux over night and then cooled to room temperature and filtered. HPLC/MS indicated the presents of product 12b.

MS (ES+) m/z: [MH]⁺=437.3.

### c) 9-Amino-1-oxo-6,7-dihydro-1H,5H-pyrido[3,2,1-ij]quinoline-2-carboxylic acid ethyl ester

To the mixture of Intermediate 12b 5% HCl was added dropwise until appearance of precipitate. Precipitate was filtered and dried in vacuum drier yielding 2 g of the crude title product.

MS (ES+) m/z: [MH]⁺=273.2

¹³C-NMR (125 MHz, DMSO) δ: 13.81, 19.90, 25.57, 51.37, 59.24, 108.39, 115.66, 124.99, 128.06, 129.06, 129.91, 130.51, 133.95, 147.54, 163.98, 171.63.

### d) 9-(2-Benzyloxy-ethylamino)-1-oxo-6,7-dihydro-1H,5H-pyrido[3,2,1-ij]quinoline-2-carboxylic acid ethyl ester

To the solution of Intermediate 12c (200 mg, 0.73 mmol) in MeOH (75 mL), benzyloxyacetaldehide (110 mg, 0.73 mmol), NaBH₃CN (137 mg, 2.2 mmol) and AcOH (250 μl) was added. Reaction mixture was stirred for 20 minutes and evaporated in vacuum. Oil product was purified by column chromatography in system CH₂Cl₂—(MeOH—NH₄OH=9:1.5)=9:(1.5) yielding 159 mg of the title product.

MS (ES+) m/z: [MH]⁺=407.2.

### e) 9-(2-hydroxy-ethylamino)-1-oxo-6,7-dihydro-1H,5H-pyrido[3,2,1-ij]quinoline-2-carboxylic acid

To the solution of Intermediate 12d (159 mg, 0.39 mmol) in EtOH (41.6 mL) cyclohexene (12.8 mL) and 10% Pd/C (243 mg) were added. The mixture was stirred under reflux over night, filtered through celite and evaporated in vacuum yielding 80 mg of the title product.

### Intermediate 13

### 6-{2-[2-(2-Carboxy-ethoxy)-ethoxy]-ethoxy}-7-chloro-1-cyclopropyl-4-oxo-1,4-dihydro-quinolone-3-carboxylic acid

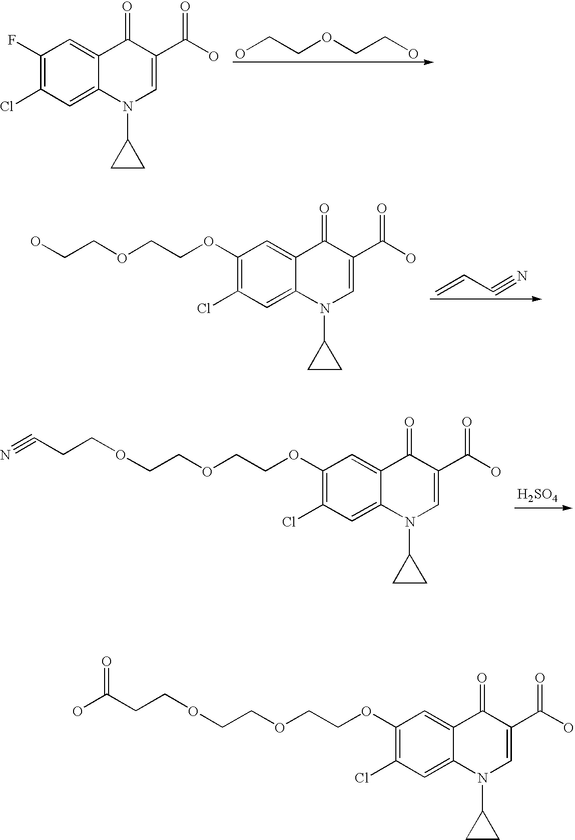

Mixture of 50 mL diethylene glycole and 50 mL DMSO was prepared and heated on 70° C. Into mixture 8 g of KO-t-Bu portionwise was added. Then, 5 g of fluoro-chloro quinolonic acid (17.8 mmol) was added portionwise. The temperature was increased to 105° C. After 5 hours, the 25 mL of H₂O was added and the mixture was extracted with 2×20 mL of DCM. Water layer was adjusted to pH 4. The obtained precipitate was filtered off and dried under reduced pressure affording 500 mg of 7-chloro-1-cyclopropyl-6-[2-(2-hydroxy-ethoxy)-ethoxy]-4-oxo-1,4-dihydro-quinolone-3-carboxylic acid.

7-Chloro-1-cyclopropyl-6-[2-(2-hydroxy-ethoxy)-ethoxy]-4-oxo-1,4-dihydro-quinolone-3-carboxylic acid (500 mg) was dissolved in 12.5 mL of acrylonitrile, then 1 mL of DBU was added and the mixture was stirred for 24 hours at 80° C. Acrylonitrile was evaporated under reduced pressure, residue was dissolved in 300 mL of 2-propanol and the pH of the mixture was adjusted to pH 3.5. The precipitate was obtained after 12 hours, filtered off and washed with water (pH 3.5). The precipitate was dissolved in 20 mL H₂O:H₂SO₄ (1:1) and stirred for 24 hours at room temperature. The obtained precipitate was filtered off and dried under reduced pressure affording 300 mg of the title compound.

### Intermediate 14

### 1-Oxo-9-(3-piperazin-1-yl)-prop-1-ynyl)-6,7-dihydro-1H,5H-pyrido[3,2,1-ij]quinoline-2-carboxylic acid ethyl ester

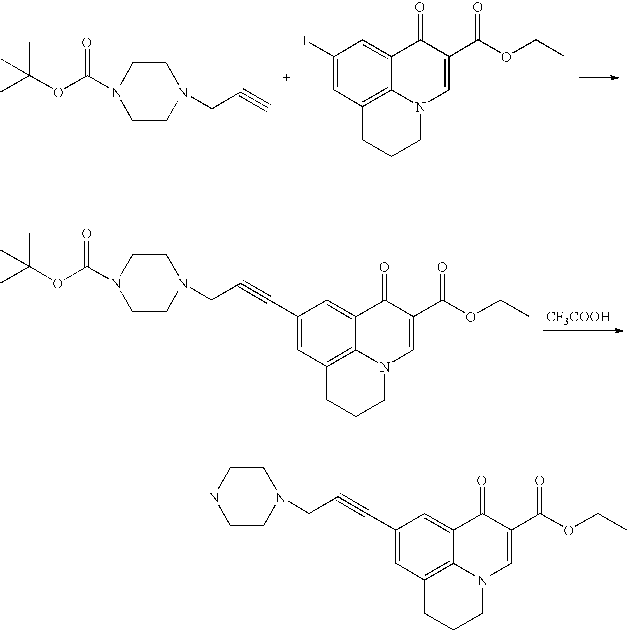

### a) 4-Prop-2-ynyl-piperazine-1-carboxylic acid tert-butyl ester

To the degassed solution of piperazine-1-carboxylic acid tert-butyl ester (0.5 g, 2.69 mmol) in acetonitrile (5 mL) was added Na₂CO₃ (0.854 g, 8.05 mmol) and mixture was stirred for 20 min. The suspension was heated to 50° C. and 3-bromo-propyne (448.65 μl, 4.03 mmol) was added. The solvent was evaporated and the residue was extracted with EtOAc and water. Organic layer was washed with NaCl and NaHCO₃ (2×20 ml), dried over K₂CO₃ and evaporated in vacuum yielding 0.45 g of the title product as yellowish oil.

MS (ES+) m/z: [MH]⁺=247.2.

### b) 9-[3-(4-tert-Butoxycarbinyl-piperazin-1-yl)-prop-1-ynyl]-1-oxo-6,7-dihydro-1H,5H-pyrido[3,2,1-ij]quinoline-2-carboxylic acid ethyl ester

Intermediate 16 (0.2 g, 0.524 mmol), copper (I) iodide (9.98 mg, 0.0524 mmol) and triethylamine (2.54 ml, 18.34 mmol) were suspended in dry acetonitrile (10 mL). The suspension was heated to 50° C. and N₂ bubbled through. After 20 min, dichlorobis (triphenylphosphine) palladium (II) (11.03 mg, 0.0157 mmol) and Intermediate 14a (0.164 g 0.733 mmol) were added and dark red suspension was heated for 3 hours at 50° C. The solvent was evaporated and the residue was extracted with EtOAc and water (2×20 ml). Organic layer was washed with NaCl and NaHCO₃ (2×20 ml), dried over K₂CO₃ and evaporated in vacuum yielding 0.34 g of the title product as red oil.

### c) 1-Oxo-9-(3-piperazin-1-yl)-prop-1-ynyl)-6,7-dihydro-1H,5H-pyrido[3,2,1-ij]quinoline-2-carboxylic acid ethyl ester

To the solution of Intermediate 14b (0.34 g, 0.71 mmol) in DCM (3.4 mL) was added CF₃COOH (3.4 mL) and mixture was stirred for 48 hours at room temp. To the reaction mixture was added water (pH 1.2) and layers were separated (pH 9.6). The organic layer was dried over K₂CO₃ and evaporated in vacuum yielding 0.22 g of the title product as red oil.

MS (ES+) m/z: [MH]⁺=380.2.

### Intermediate 15

### 10-Amino-1-Oxo-6,7-dihydro-1H,5H-pyrido[3,2,1-ij]quinoline-2-carboxylic acid ethyl ester

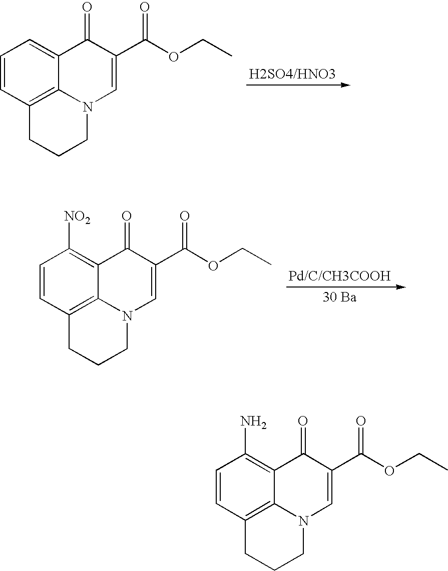

### a) 10-Nitro-1-Oxo-6,7-dihydro-1H,5H-pyrido[3,2,1-ij]quinoline-2-carboxylic acid ethyl ester

1-Oxo-6,7-dihydro-1H,5H-pyrido[3,2,1-ij]quinoline-2-carboxylic acid ethyl ester (1.0 g) was placed in round bottom flask and to that, mixture of H₂SO₄/HNO₃ (1:1) was added and stirred for 3 hours at 0° C. The reaction mixture was poured on ice and precipitate was filtered off affording 900 mg of title product (LC/MS: 95%).

### b) 10-Amino-1-Oxo-6,7-dihydro-1H,5H-pyrido[3,2,1-ij]quinoline-2-carboxylic acid ethyl ester

Intermediate 15a (9oo mg)was diluted in 35 mL of acetic acid and to this mixture 800 mg of 10% Pd/C was added and stirred for 15 h at room temperature and at 30 Ba. The reaction mixture was filtered to remove catalyst and then acetic acid was evaporated under reduced pressure affording 700 mg of the title product. (LC/MS: 95%).

### Intermediate 16

### 9-Iodo-1-oxo-6,7-dihydro-1H,5H-pyrido[3,2,1-ij]quinoline-2-carboxylic acid ethyl ester

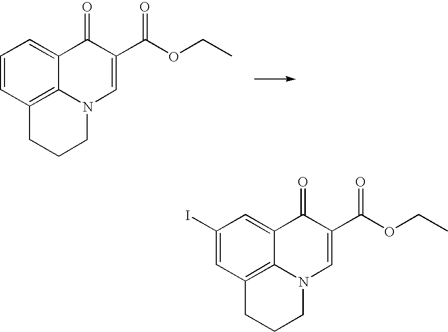

To a 0° C. cooled trifluoromethanesulfonic acid (3 mL, 33.31 mmol) 1-Oxo-6,7-dihydro-1H,5H-pyrido[3,2,1-ij]quinoline-2-carboxylic acid ethyl ester (1.53 g, 5.95 mmol) was added and to that solution N-Iodosuccinimide (1.6 g, 714 mmol) was added. The mixture was allowed to warm from 0° C. to room temperature while stirring. Reaction mixture was poured in ice and precipitate was filtered off affording 1 g of the title product (LC/MS: 57%).

### Intermediate 17

### 3-(2-tert-butoxycarbonylethyl)-imidazolidine-1-carboxylic acid tert-butyl ester

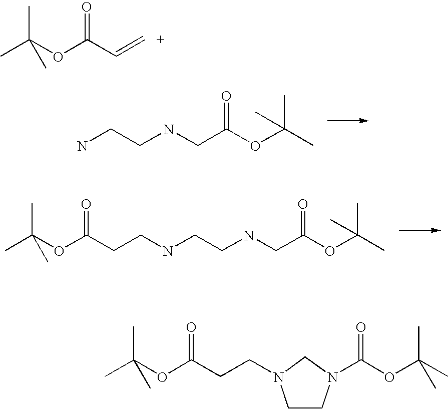

### a) 3-[2-(tert-butoxycarbonylmethyl-amino)-ethylamino]-propionic acid tert-butyl ester

To the solution of (2-amino-ethylamino)-acetic acid tert-butyl ester (1.0 mL, 6.32 mmol) in

i-PrOH (50 mL) was added acrylic acid tert-butyl ester (309.1 μL, 2.11 mmol). The suspension was heated for 48 hours at 60° C. The solvent was evaporated and product was purificated by column chromatography (DCM-MeOH—NH₃=90:3:0.5) yielded the title product as colorless oil (0.45 mg).

MS (ES+) m/z: [MH]⁺=289.2

### b) 3-(2-tert-butoxycarbonylethyl)-imidazolidine-1-carboxylic acid tert-butyl ester

To the solution of Intermediate 17a (0.45 mg, 1.56 mmol) in chloroform (20 mL) were added HCOOH (0.218 mL, 5.78 mmol) and HCHO (0.24 mL, 8.69 mmol) and stirred at room temperature for 2 hours. To the reaction mixture was added water (pH 1.3) and layers were separated (pH 2.5). The organic layer was dried over K₂CO₃ and evaporated in vacuum yielding 034 g of oil colorless product.

MS (ES+) m/z: [MH]⁺=301.2

### Intermediate 18

### 6-Amino-1-ethyl-4-oxo-1,4-dihydro-quinoline-3-carboxylic acid ethyl ester

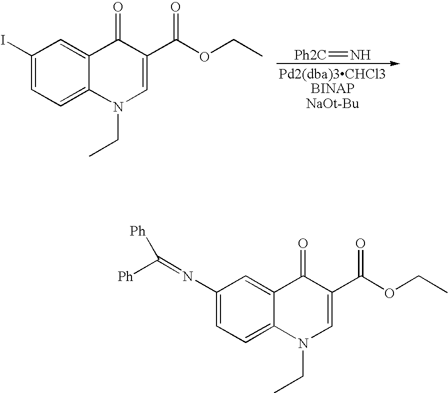

### a) 6-(Benzhydrylidene-amino)-1-ethyl-4-oxo-1,4-dihydro-quinoline-3-carboxylic acid ethyl ester

A Pyrex tube was charged with sodium tert-butoxide (1.4 mmol), Pd₂(dba)₃ (0.00125 mmol), and BINAP (0.00375 mmol). The Pyrex tube was fitted with a septum and after the air atmosphere was replaced with argon, toluene (4 mL), 1-ethyl-6-iodo-4-oxo-1,4-dihydro-quinoline-3-carboxylic acid ethyl ester (1.0 mmol), and benzophenone imine (1.2 mmol) were added by syringe. The reaction was sealed and heated to 80° C. with stirring until starting material was consumed as judged by GC analysis. The reaction mixture was cooled to room temperature, diluted with ether (40 mL), filtered, and concentrated. The crude reaction mixture was then recrystallized from MeOH to furnish the desired product in 90% yield.

### b) 6-Amino-1-ethyl-4-oxo-1,4-dihydro-quinoline-3-carboxylic acid ethyl ester

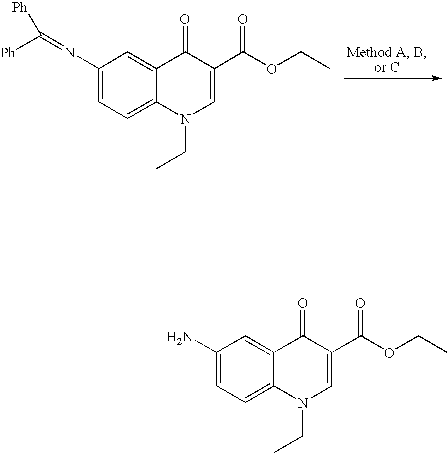

Method A: Transamination with Hydroxylamine

To a solution of the imine adduct in MeOH (0.1 M) at RT was added NaOAc (2.4 eq) and hydroxylamine hydrochloride (1.8 eq). Oxime formation was usually complete in 15 to 30 minutes. The solution was then partitioned between 0.1 M NaOH and CH₂Cl₂. The organic layer was dried over anhydrous Na₂SO₄ and concentrated in vacuo. The product was purified by chromatography on silica gel.

### Method B: Hydrogenolysis

A solution of the imine adduct, ammonium formate (15 equiv.) and 5% Pd/C (10 mol %) was heated to 60° C. in MeOH (0.2 M in imine). After 2 hours, reduction was usually complete. The solution was cooled to room temperature and diluted with CH₂Cl₂ (5× volume of MeOH) to be passed through a plug of celite. The organic solution was washed with 0.1 M NaOH, dried over anhydrous Na₂SO₄ and concentrated in vacuo. The product was purified by chromatography on silica gel.

### Method C: Acidic Hydrolysis

To a solution of the imine adduct in THF (0.3 M) aqueous 2.0 M HCl (added 5% by volume of THF) was added. After 5-20 minutes hydrolysis was complete and the reaction mixture was partitioned between 0.5 M HCl and 2:1 hexane/EtOAc. The aqueous layer was separated and made alkaline. The product aniline was extracted with CH₂Cl₂, dried over anhydrous Na₂SO₄ and concentrated in vacuo.

### Intermediate 19:

### 4″-Hydroxyimino azithromycin

4″-Keto azithromycin (5.2 g, 0.007 mol) was treated with hydroxylamine hydrochloride (2.4 g) in MeOH (260 ml) for 3.5 hours at room temperature. The methanol was evaporated and the residue dissolved in EtOAc (200 ml). Water was then added (200 ml) and extracted at pH 9.8. Solvent was removed affording crude product (5.39 g). After purification by flash chromatography (DCM-MeOH—NH₄OH=90:9:0.5) the title compound (2.4 g) was obtained;

MS (ES+) m/z: [MH]⁺=762.33.

### Intermediates 20:

### 4″-(S) Amino azithromycin (A) and

### 4″-(R)-Amino azithromycin (B)

### Method I

Intermediate 19 (2.0 g, 0.0026 mol) was dissolved in acetic acid (100 mL) and hydrogenated over 2.0 g platinum oxide at 1150 psi for 48 hours at room temperature. This was followed by a fresh addition of 0.8 g of platinum oxide and the reaction was continued for another 24 hours under 1150 psi. Since TLC shown some starting compound a further 0.8 g platinum oxide was added and reduction continued for a further 24 hours at the same pressure. The reaction mixture was filtered and acetic acid was removed under vacuum. The residue was dissolved in 100 mL of CHCL₃ and 50 mL of water and extracted at pH 5 and 10. Evaporation of the extract at pH 10 afforded a mixture of 4″-(S) and 4″-(R) amines (1.96 g).

After purification by column chromatography (DCM-MeOH—NH₄OH=90:9:1.5) two separate isomers were isolated: Intermediate 20 A with Rf=0.67, δ 4.10, dq, H-5″, 4″-(S)-amine and Intermediate 20 B with Rf=0.63, δ 4.57, dq, H-5″,4″-(R)-amine.

MS (ES+) m/z: [MH]⁺=748.36.

### 4″-(S) Amino azithromycin (A)

¹³C-NMR (125 MHz, CDCl₃) δ: 178.8, 102.9, 94.9, 83.9, 77.8, 77.4, 74.3, 73.8, 73.6, 72.7, 70.9, 70.1, 68.4, 66.7, 65.7, 62.5, 62.0, 49.4, 45.2, 42.4, 41.9, 40.4, 36.3, 35.2, 29.1, 27.5, 26.8, 22.5, 22.0, 21.5, 21.3, 18.9, 16.2, 14.7, 11.3, 9.2, 7.5. and

### 4″-(R)-Amino azithromycin (B)

¹³C-NMR (125 MHz, CDCl₃) δ: 179.0, 102.7, 95.5, 83.8, 78.3, 77.4, 74.7, 74.1, 73.6, 73.4, 71.0, 70.2, 68.2, 65.6, 63.0, 62.5, 58.2, 49.3, 45.5, 42.4, 42.3, 40.4, 36.2, 29.4, 27.4, 26.7, 22.1, 22.1, 21.4, 21.3, 18.5, 16.3, 15.1, 11.4, 9.4, 7.2.

### Method II

### 4″-(R)-Amino azithromycin (B)

To a solution of NaOAc (2.0 g, 24.4 mmol) in MeOH (42 ml), cooled to 0° C. Intermediate 19 (1.25 g, 1.6 mmol) was added. To the reaction solution NaCNBH₃ (0.267 g, 4.0 mmol) was added portionwise during 20 min, followed by addition of TlCl₃ (3.8 ml, 10% solution of TiCL₃ in 20% HCl) during 30 min while maintaining temperature at 0° C. The reaction mixture was stirred for further 30 minutes at the same temperature, and than poured in ice-H₂O mixture (100 ml). The pH of the reaction mixture was adjusted to 10.5 and extracted with EtOAc (2×70 ml). The combined organic layers were dried over K₂CO₃ and concentrated in vacuo. The residue was suspended in diisopropylether (2×50 ml), filtered off and the filtrate concentrated in vacuo affording 1:1 mixture (990 mg) of starting oxime and the title product as a white foam which was used in further reactions without separation. If desired the obtained mixture may be separated by column chromatography (solvent system: DCM/MeOH/NH₄OH=90:9:1.5).

### Intermediate 21

### 7-Chloro-1-cyclopropyl-6-(2-hydroxy-ethoxy)-4-oxo-1,4-dihydro-quinoline-3-carboxylic acid (A) and

### 1-Cyclopropyl-6-fluoro-7-(2-hydroxy-ethoxy)-4-oxo-1,4-dihydro-quinoline-3-carboxylic acid (B)

To a mixture of DMSO (5 mL) and ethyleneglycol (6 mL), KOtBu (1.6 g, 14.23 mmol) was added portionwise over 10 min, and then heated to 90° C. To the mixture, 7-chloro-1-cyclopropyl-6-fluoro-4-oxo-1,4-dihydro-quinoline-3-carboxylic acid (1.0 g) was added portionwise over 20 min, the temperature was increased to 105° C. and the mixture was stirred for 6 h. Water (30 mL) was added to the reaction solution and the pH of the solution was adjusted to pH=5. The resulting solution was left in the refrigerator overnight. The precipitate obtained was filtered, washed with cold water, and dried affording a 2:1 mixture of Intermediate 21A and Intermediate 21B (1.0 g).

Part of the crude product (700 mg) was dissolved in EtOH (15 mL) by heating to the reflux. The resulting solution was cooled to 30° C. and a first precipitation occurred. The precipitate was filtered, washed with cold EtOH and dried under reduced pressure. Intermediate 21A (204 mg) was obtained as a white solid;

¹H-NMR (500 MHz, DMSO-d6) δ: 15.06 (s, 1H), 8.71 (s, 1H), 8.40 (s, 1H), 7.86 (s, 1H), 4.97 (t, 1H), 4.25 (t, 2H), 3.87 (m, 1H), 3.82 (q, 2H), 1.32 (m, 2H), 1.20 (m, 2H); ¹³C-NMR (75 MHz, DMSO-d6) δ: 176.61, 165.67, 152.47, 147.54, 135.34, 129.48, 124.95, 120.02, 106.90, 106.66, 71.22, 59.15, 35.99, 7.46;

### Intermediate 22

### 7-{2-[2-(2-carboxy-ethoxy)ethoxy]ethylamino}-1-cyclopropyl-6-fluoro-4-oxo-1,4-dihydro-quinoline-3-carboxylic acid (A) and

### 6-{2-[2-(2-carboxy-ethoxy)ethoxy]ethylamino}-1-cyclopropyl-7-chloro-4-oxo-1,4-dihydro-quinoline-3-carboxylic acid (B)

Using the similar procedure to that described in Intermediate 7, starting from a mixture of Intermediate 6A and Intermediate 6B, Intermediate 27 was prepared as a mixture of Intermediate 27A and Intermediate 22B in a 1:1 ratio.

### Intermediate 23

### 6-{2-[2-(2-amino-ethoxy)-ethoxy]-ethylamino}-1-cyclopropyl-7-chloro-4-oxo-1,4-dihydro-quinoline-3-carboxylic acid (A) and

### 7-{2-[2-(2-amino-ethoxy)-ethoxy]-ethylamino}-1-cyclopropyl-6-fluoro-4-oxo-1,4-dihydro-quinoline-3-carboxylic acid (B)

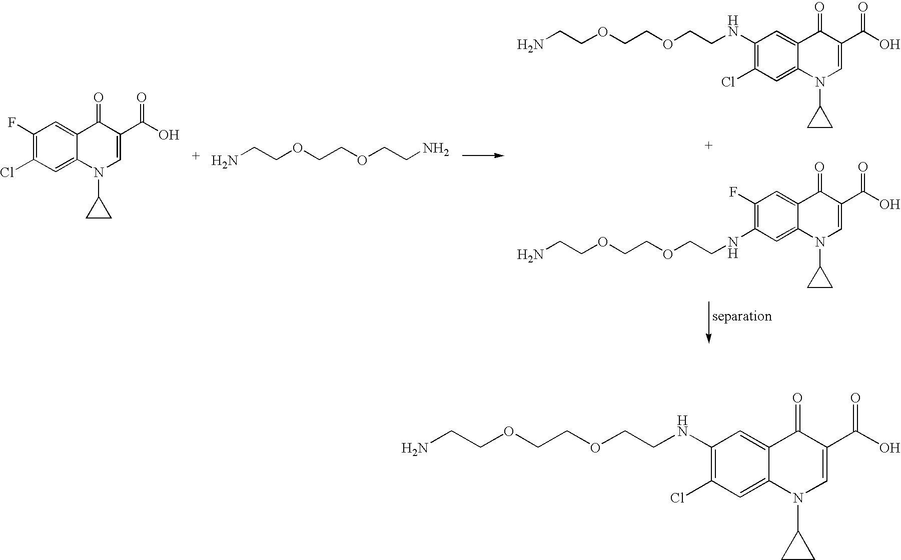

A mixture of 7-chloro-1-cyclopropyl-6-fluoro-4-oxo-1,4-dihydro-quinoline-3-carboxylic acid (5 g, 0.018 mol), 2,2′-(ethylenedioxy)bis-(ethylamine) (26 mL, 0.18 mol, 10 eq.) in 1-methyl-2-pyrrolidone was heated at 110° C. for 24 hours. Reaction mixture was diluted with water (70 mL) pH was adjusted to 11 and extracted with CH₂Cl₂ (9×40 mL). Water layer was then acified to pH 6.8 with H₂SO₄, extracted with CH₂Cl₂ (50 mL) and evaporated. 2-Propanol was added (200 mL) and stirred at 82° C. for 30 minutes. The reaction mixture was then filtered and 2-propanol was evaporated in vacuum yielding 8 g of oily product, according to LC-MS 50% of chloro derivative (A) and 30% of fluoro derivative. Product was purified by column chromatography (eluent CH₂Cl₂-2-propanol=1:1) yielding pure chloro derivative (A).

MS (ES+) m/z: [MH]⁺=409.9 (A)

MS (ES+) m/z: [MH]⁺=393.4 (B)

### Intermediate 24

### 6-[2-(2-Amino-ethoxy)-ethylamino]-7-chloro-1-cyclopropyl-4-oxo-1,4-dihydro-quinoline-3-carboxylic acid (A) and

### 7-[2-(2-Amino-ethoxy)-ethylamino]-1-cyclopropyl-6-fluoro-4-oxo-1,4-dihydro-quinoline-3-carboxylic acid (B)

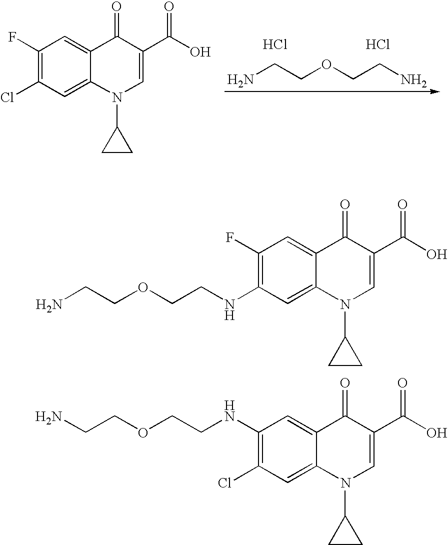

To a solution of 7-chloro-1-cyclopropyl-6-fluoro-4-oxo-1,4-dihydro-quinoline-3-carboxylic acid (0.55 g, 1.95 mmol) in 1-methyl-2-pyrrolidone (40 mL) bis-(2-aminoethyl)-ether dihydrochlorid (2.1 g, 11.9 mmol, 6 eq.) and DBU (3.49 mL, 23.4 mmol, 12 eq.) added and the reaction mixture was stirred at 110° C. for 18 hours. The reaction mixture was then diluted with water (70 mL), pH was adjusted to 11 and extracted with CH₂Cl₂ (9×40 mL). Water layer was then acified with H₂SO₄ to pH 6.8, extracted with 50 mL of CH₂Cl₂ and then evaporated in vacuum. Crude product was diluted in 2-propanol (60 mL), stirred at 82° C. for 20 minutes and filtrated. Precipitate was pure salt (Na₂SO₄). 2-Propanol was evaporated in vacuum and product was purified by column chromatography (fraction, eluent: CH₂Cl₂—MeOH—NH₃—CH₃CN=4:4:2:1) yielding 0.5 g of title compounds as a mixture of chloro and fluoro derivatives in ratio 3:1

MS (ES+) m/z: [MH]⁺=365.8 (A) (75%)

MS (ES+) m/z: [MH]⁺=349.4 (B) (25%)

### Intermediate 25

### 6-{3-[2-(2-Carboxy-ethoxy)-ethoxy]-propyl}-1-dimethylamino-4-oxo-1,4-dihydro-quinoline-3-carboxylic acid

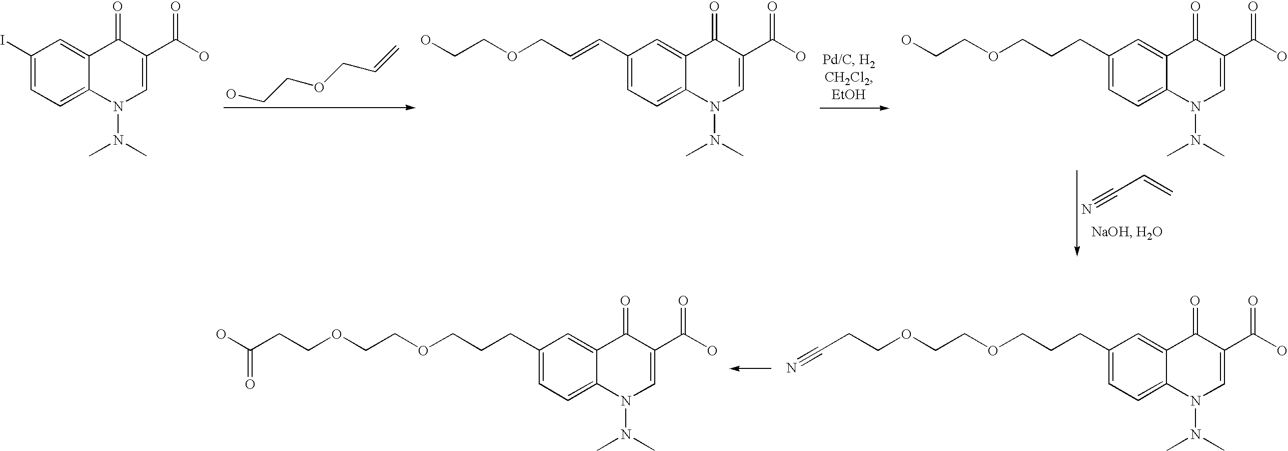

### a) 1-Dimethylamino-6-[3-(2-hydroxy-ethoxy)-propenyl]-4-oxo-1,4-dihydro-quinoline-3-carboxylic acid

To a solution of 2-allyloxy-ethanol (6.3 ml, 59.53 mmol, 6 eq.) in DMF (46 ml), Pd(OAc)₂ (0.103 g, 0.46 mmol) was added under N₂ atmosphere. After 1 hour 1-dimethylamino-6-iodo-4-oxo-1,4-dihydro-quinoline-3-carboxylic acid (3.28 g, 9.16 mmol) and Et₃N (3.2 ml, 33 mmol, 2.5 eq.) were added. Than N₂ atmosphere was removed and the reaction mixture was stirred at 60° C. for 1 h and for further 18 hours at 75° C. Catalyst was filtered off, H₂O (130 ml) and DCM (65 ml) were added and pH adjusted to 12.5. The H₂O layer was extracted with DCM (2×50 ml). pH of the H₂O layer was adjusted to pH 7.3 during which precipitation occurred. The precipitate was filtered off, washed with H₂O, dried at 40° C. for 18 h, affording the title product (2.27 g).

MS (ES+) m/z: [MH]⁺=333.1.

### b) 1-Dimethylamino-6-[3-(2-hydroxy-ethoxy)-propyl]-4-oxo-1,4-dihydro-quinoline-3-carboxylic acid

To a suspension of Intermediate 25a (2.27 g, 6.83 mmol) in DCM (40 ml) and EtOH (90 ml), 10% Pd/C (0.513 g) was added and obtained mixture hydrogenated at 4 bar of H₂-pressure for 18 hours. The catalyst was filtered off, washed with DCM and the solvent evaporated in vacuo. Crude product was precipitated from DCM/diisopropyl ether affording the title product (2.1 g).

MS (ES+) m/z: [MH]⁺=335.1.

### c) 6-{3-[2-(2-Cyano-ethoxy)-ethoxy]-propyl}-1-dimethylamino-4-oxo-1,4-dihydro-quinoline-3-carboxylic acid

Suspension of Intermediate 25b (1.55 g, 4.6 mmol) in MIBC (17 ml) and 40% NaOH (0.97 ml, 13.9 mol) was cooled to 0-5° C. After 10 minutes of stirring acrylonitrile (5.2 mL, 79.0 mmol) was added dropwise and reaction mixture was further stirred at 15-20° C. for 2 hours. To the reaction mixture water (10 ml) was added, suspension was filtered and layers were separated. Organic layer was washed with water (10 ml). Combined water layers were acidified to pH 6, during which precipitation occurred. Precipitate was filtered off, washed with water and dried affording the title product (1.51 g).

MS (ES+) m/z: [MH]⁺=388.1.

### d) 6-{3-[2-(2-Carboxy-ethoxy)-ethoxy]-propyl}-1-dimethylamino-4-oxo-1,4-dihydro-quinoline-3-carboxylic acid

To a concentrated H₂SO₄ (10 mL) cooled to 0-5° C., Intermediate 25 c (1.44 g, 3.72 mmol) was added portionwise while maintaining temperature at 0-5° C. The reaction mixture was stirred at 10-20° C. for 1 hour and then at 20-22° C. for further 18 hours. The reaction mixture was added drop-wise to water (21.5 ml) while maintaining temperature at 0-5° C. and stirred at 70-80° C. for 24 hours. Than the reaction mixture was cooled at 0-5° C., water (12 ml) was added, the pH was adjusted to 2.3, during which precipitation occurred. The precipitate was filtered off, washed with water and dried at 60° C. under reduced pressure affording the title product (1.22 g).

MS (ES+) m/z: [MH]⁺=407.0.

### Intermediate 26

### 6-{3-[2-(2-Carboxy-ethoxy)-ethoxy]-propyl}-1-(2-methoxy-ethyl)-4-oxo-1,4-dihydro-quinoline-3-carboxylic acid

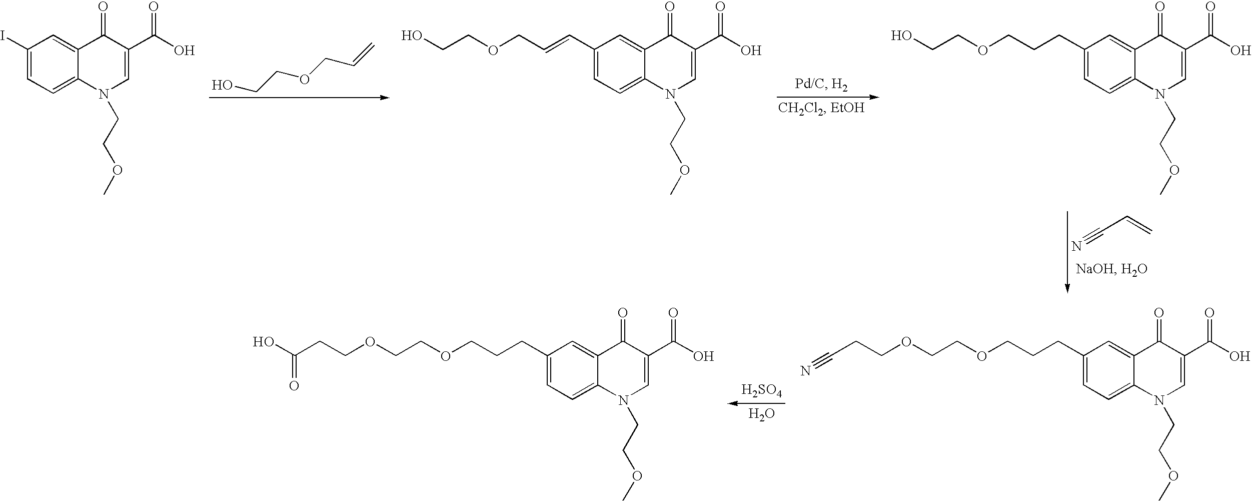

### a) 6-[3-(2-Hydroxy-ethoxy)-propenyl]-1-(2-methoxy-ethyl)-4-oxo-1,4-dihydro-quinoline-3-carboxylic acid

According to the procedure of Intermediate 25a starting from 1-(2-methoxy-ethyl)-6-iodo-4-oxo-1,4-dihydro-quinoline-3-carboxylic acid (5.0 g, 14.4 mmol) and 2-allyloxy-ethanol (9.3 ml, 87.1 mmol) the title compound was obtained (3.53 g).

MS (ES+) m/z: [MH]⁺=348.1.

### b) 6-[3-(2-Hydroxy-ethoxy)-propyl]-1-(2-methoxy-ethyl)-4-oxo-1,4-dihydro-quinoline-3-carboxylic acid

According to the procedure of Intermediate 25b starting from Intermediate 26a (3.42 g, 9.9 mmol) the title compound was obtained (2.77 g).

MS (ES+) m/z: [MH]⁺=350.1.

### c) 6-{3-[2-(2-Cyano-ethoxy)-ethoxy]-propyl}-1-(2-methoxy-ethyl)-4-oxo-1,4-dihydro-quinoline-3-carboxylic acid

According to the procedure of Intermediate 25c starting from Intermediate 26b (2.77 g, 7.9 mmol) and acrylonitrile (8.9 ml, 134 mmol) the title compound was obtained (2.71 g).

MS (ES+) m/z: [MH]⁺=403.1.

### d) 6-{3-[2-(2-Carboxy-ethoxy)-ethoxy]-propyl}-1-(2-methoxy-ethyl)-4-oxo-1,4-dihydro-quinoline-3-carboxylic acid

According to the procedure of Intermediate 25d starting from Intermediate 26c (2.65 g, 6.5 mmol) the title compound was obtained (2.62 g).

MS (ES+) m/z: [MH]⁺=422.1.

### Intermediate 27

### 6-{3-[2-(2-Carboxy-ethoxy)-ethoxy]-propyl}-1-cyclopropyl-4-oxo-1,4-dihydro-quinoline-3-carboxylic acid

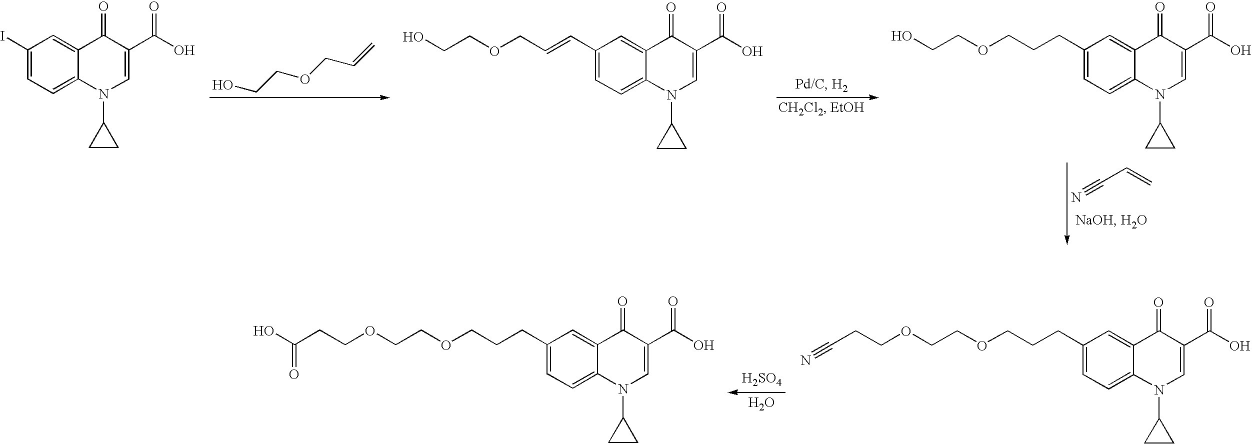

### a) 1-Cyclopropyl-6-[3-(2-hydroxy-ethoxy)-propenyl]-4-oxo-1,4-dihydro-quinoline-3-carboxylic acid

According to the procedure of Intermediate 25a starting from 1-cyclopropyl-6-iodo-4-oxo-1,4-dihydro-quinoline-3-carboxylic acid (15.0 g, 42.2 mmol) and 2-allyloxy-ethanol (29.4 ml, 275 mmol) the title compound was obtained (8.05 g).

MS (ES+) m/z: [MH]⁺=330.1.

### b) 1-Cyclopropyl-6-[3-(2-hydroxy-ethoxy)-propyl]-4-oxo-1,4-dihydro-quinoline-3-carboxylic acid

According to the procedure of Intermediate 25b starting from Intermediate 27a (8 g, 24.2 mmol) the title compound was obtained (6.85 g).

MS (ES+) m/z: [MH]⁺=332.1

¹³C-NMR (75 MHz, DMSO) δ: 178.6; 166.8; 148.8; 141.3; 140.2; 135.7; 125.7; 125.1; 119.3; 107.9; 70.4; 70.2; 70.1; 66.1; 31.9; 36.6; 31.5; 18.9; 8.3.

### c) 6-{3-[2-(2-Cyano-ethoxy)-ethoxy]-propyl}-1-cyclopropyl-4-oxo-1,4-dihydro-quinoline-3-carboxylic acid 1,4-dihydro-quinoline-3-carboxylic acid

According to the procedure of Intermediate 25c starting from Intermediate 27b (6.85 g, 20.7 mmol) and acrylonitrile (23.2 ml, 351 mmol) the title compound was obtained (7.05 g).

MS (ES+) m/z: [MH]⁺=385.1.

### d) 6-{3-[2-(2-Carboxy-ethoxy)-ethoxy]-propyl}-6-cyclopropyl-4-oxo-1,4-dihydro-quinoline-3-carboxylic acid

According to the procedure of Intermediate 25d starting from Intermediate 27c (6.98 g, 6.5 mmol) the title compound was obtained (5.95 g).

MS (ES+) m/z: [MH]⁺=404.1.

### Intermediate 28

### 6-{3-[2-(2-Carboxy-ethoxy)-ethoxy]-propyl}-1-ethyl-4-oxo-1,4-dihydro-quinoline-3-carboxylic acid

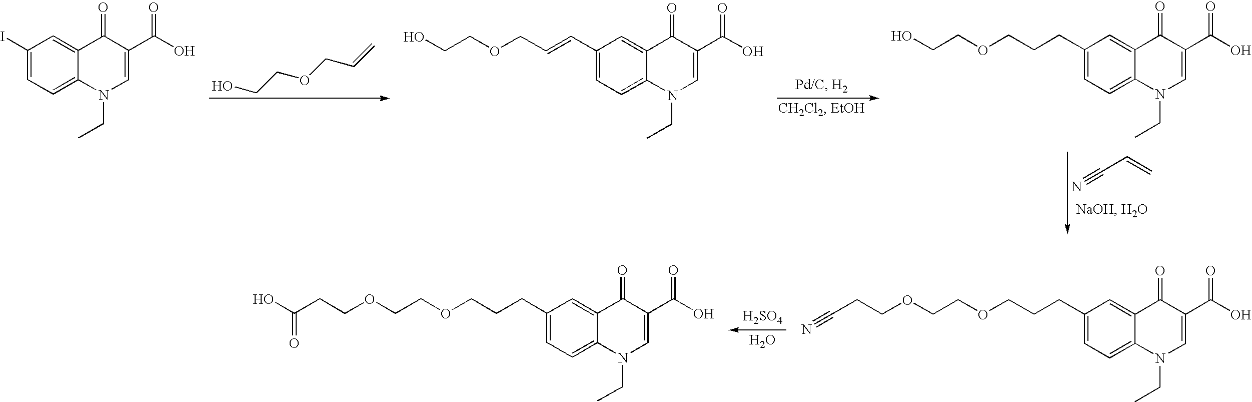

### a) 1-Ethyl-6-[3-(2-hydroxy-ethoxy)-propenyl]-4-oxo-1,4-dihydro-quinoline-3-carboxylic acid

According to the procedure of Intermediate 25a starting from 1-ethyl-6-iodo-4-oxo-1,4-dihydro-quinoline-3-carboxylic acid (5.0 g, 14.6 mmol) and 2-allyloxy-ethanol (5.0 ml, 46.0 mmol) the title compound was obtained (2.85 g).

MS (ES+) m/z: [MH]⁺=318.1.

### b) 1-Ethyl-6-[3-(2-hydroxy-ethoxy)-propyl]-4-oxo-1,4-dihydro-quinoline-3-carboxylic acid

According to the procedure of Intermediate 25b starting from Intermediate 28a (2.8 g, 8.8 mmol) the title compound was obtained (2.2 g).

MS (ES+) m/z: [MH]⁺=320.1

¹³C-NMR (75 MHz, DMSO) δ: 177.6; 166.3; 148.7; 140.5; 137.5; 135.1; 125.7; 124.7; 118.2; 108.; 72.2; 69.4; 60.4; 49.1; 31.2; 30.9; 14.8.

### e) 6-{3-[2-(2-Cyano-ethoxy)-ethoxy]-propyl}-1-ethyl-4-oxo-1,4-dihydro-quinoline-3-carboxylic acid 1,4-dihydro-quinoline-3-carboxylic acid

According to the procedure of Intermediate 25c starting from Intermediate 28b (2.2 g, 7.0 mmol) and acrylonitrile (7.9 ml, 119 mmol) the title compound was obtained (2.3 g).

MS (ES+) m/z: [MH]⁺=373.2

¹³C-NMR (75 MHz, DMSO-d6) δ: 175.4, 167.2, 146.7, 137.2, 137.0, 132.6, 128.1, 125.1, 119.3, 116.5, 110, 69.6, 69.3, 69.3, 65.2, 47.3, 31.0, 30.8, 14.4.

### d) 6-{3-[2-(2-Carboxy-ethoxy)-ethoxy]-propyl}-1-ethyl-4-oxo-1,4-dihydro-quinoline-3-carboxylic acid

According to the procedure of Intermediate 25d starting from Intermediate 28c (2.3 g, 6.0 mmol) the title compound was obtained (1.3 g).

MS (ES+) m/z: [MH]⁺=392.2

¹³C-NMR (75 MHz, DMSO-d6) δ: 177.4, 172.5, 166.1, 148.4, 140.3, 137.3, 134.9, 125.4, 124.5, 118.0, 107.4, 69.5, 69.3, 69.2, 66.2, 48.9, 34.7, 31.0, 30.5, 14.5.

### Example 1

### 4″(R)+(S)—N-(3-{2-[2-(3-Carboxy-7-chloro-1-cyclopropyl-4-oxo-1,4-dihydro-quinolin-6-ylamino)-ethoxy]-ethoxy}-propionyl}-azithromycin

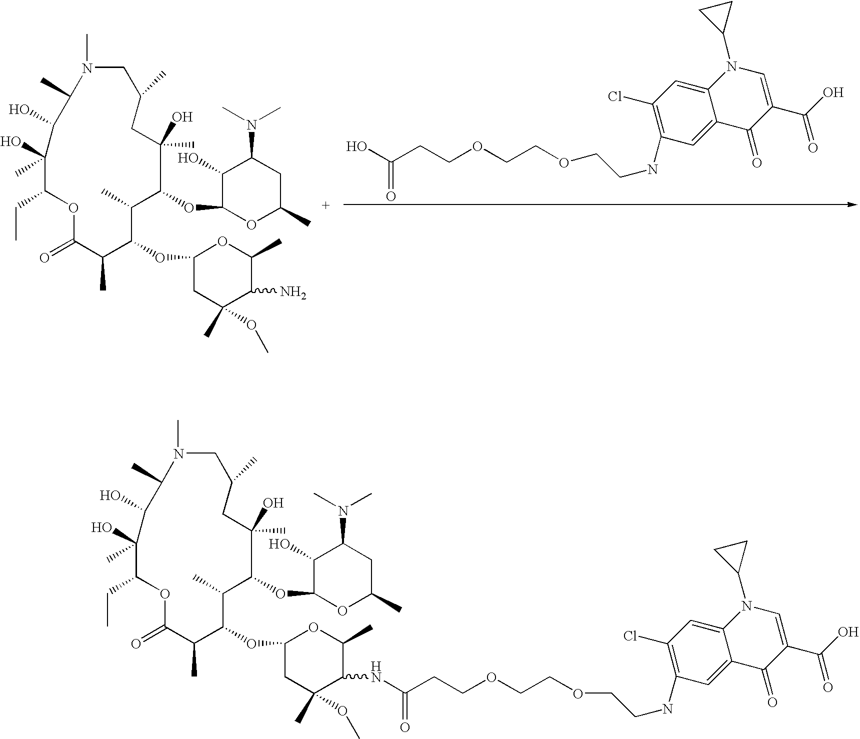

Intermediate 20 (0.073 g, 0.094 mmol, mixture of both epimers at C/4″ was dissolved in dry DCM (2.6 mL). To the reaction solution DCC (0.052 g, 026 mmol), DMAP (0.0065 g, 0.05 mmol) and Intermediate 7 (0.08 g, 0.18 mmol) were added and the reaction mixture was stirred at room temperature over night. Filtration and evaporation of solvent yielded a crude product. Purification by column chromatography (DCM-MeOH—NH₃=90:9:1.5) yielded 0.077 g of the title product. LC/MS Analysis gave % area 35.91% of the one isomer and 46.82% of the other.

MS m/z ˜1168.60 (MH+);

(M+2H)²⁺ m/z=584.80.

### Example 2

### 4″(R)—N-(3-{2-[2-(3-Carboxy-7-chloro-1-cyclopropyl-4-oxo-1,4-dihydro-quinolin-6-ylamino)-ethoxy]-ethoxy}-propionyl}-azithromycin

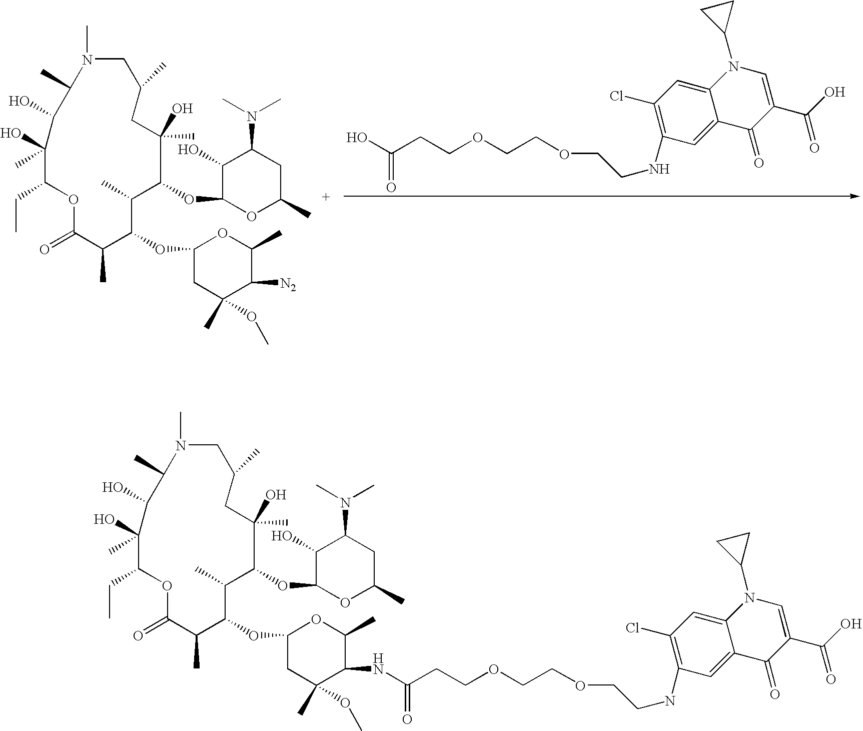

Starting from Intermediate 20B (0.1 g, 0.13 mmol) and Intermediate 7 (0.11 g) crude title compound was obtained according to the method of Example 1. Purification by column chromatography (DCM-MeOH—NH₃=90:9:1.5) yielded 0.11 g of the title product.

### Example 3

### 4″(S)—N-(3-{2-[2-(3-Carboxy-7-chloro-1-cyclopropyl-4-oxo-1,4-dihydro-quinolin-6-ylamino)-ethoxy]-ethoxy}-propionyl}-azithromycin

Starting from Intermediate 20A (0.4 g, 0.54 mmol) and Intermediate 7 (0.85 g) crude title compound was obtained according to the method of Example 1. Purification by column chromatography (DCM-MeOH—NH₃=90:9:1.5) yielded the title product.

### Example 4

### 4″(R)—N-(3-{2-[3-(3-Carboxy-1-ethyl-4-oxo-1,4-dihydro-quinolin-6-yl)-propoxy]-ethoxy}-propionyl)-azithromycin

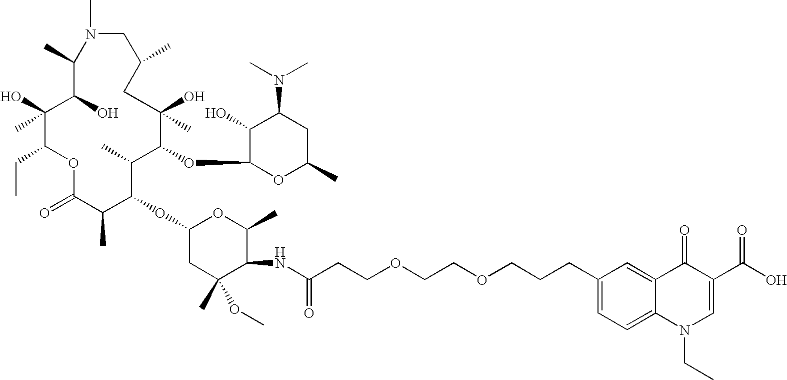

To a solution of Intermediate 28d (179 mg, 0.45 mmol) in dry DCM (10 ml) TEA (0.617 ml, 10 eq) and HOBT (119 mg, 2 eq) ware added at room temperature, followed by addition of solution of Intermediate 20B (330 mg, 1:1 mixture of amine and oxime) in DCM (1 ml). Than EDC×HCl (338 mg, 4 equiv.) was added and the mixture was stirred at room temperature overnight. Water (20 ml) was added, organic layer separated and evaporated. The crude residue was precipitated from DCM (0.5 ml) and DIPE (4 ml). The resulting precipitate was filtered off and purified by column chromatography using solvent system: DCM/MeOH/NH₃=90:9:0.5 and precipitated from EtOAc/n-hexane affording the title product (120 mg).

MS m/z: (ES): MH⁺=1121.5

¹³C NMR (125 MHz, DMSO)/δ: 177.4, 176.9, 170.7, 166.1, 148.5, 140.3, 137.3, 134.9, 125.4, 124.5, 118.0, 107.4, 102.0, 94.7, 82.7, 77.1, 77.0, 76.2, 74.9, 73.5, 73.5, 72.5, 70.1, 69.4, 69.2, 68.6, 67.1, 67.1, 64.8, 61.6, 61.4, 54.2, 49.0, 48.9, 44.6, 41.8, 41.6, 40.1, 36.0, 35.7, 30.9, 30.6, 29.0, 29.1, 27.3, 26.0, 22.0, 21.1, 21.1, 20.9, 17.7, 17.5, 14.6, 14.5, 10.8, 8.9, 6.9.

### Example 5

### 4″-(R)—N-(3-{2-[3-(3-Carboxy-1-dimethylamino-4-oxo-1,4-dihydro-quinolin-6-yl)-propoxy]-ethoxy}-propionyl)-azithromycin

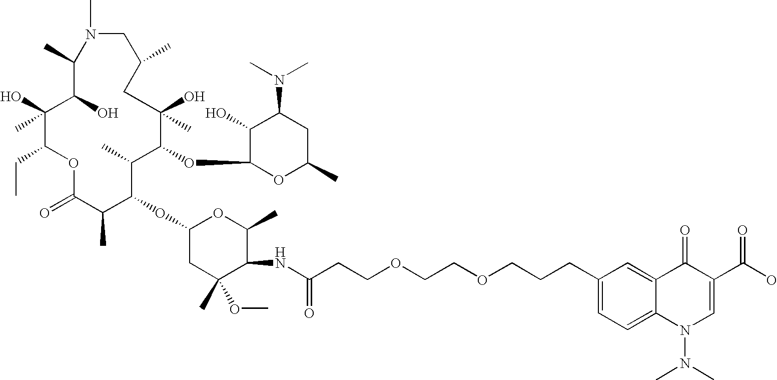

According to the procedure of Example 4 starting from Intermediate 20B (250 mg, 1:1 mixture of amine and oxime) and Intermediate 25d (136 mg, 0.33 mmol) the title compound was obtained (60 mg).

MS m/z: (ES): MH⁺=1136.4.

### Example 6

### 4″-(R)—N-(3-{2-[3-(3-Carboxy-1-(2-methoxy-ethyl-4-oxo-1,4-dihydro-quinolin-6-yl)-propoxy]-ethoxy}-propionyl)-azithromycin

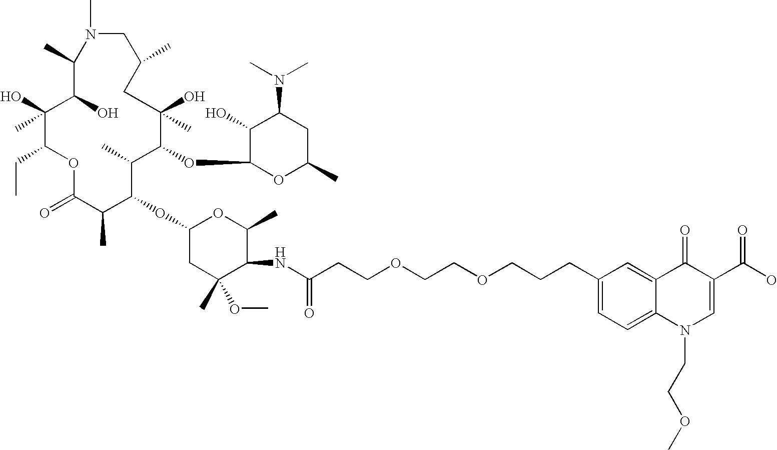

According to the procedure of Example 4 starting from Intermediate 20B (250 mg, 1:1 mixture of amine and oxime) and Intermediate 26d (141 mg, 0.33 mmol) the title compound was obtained (107 mg).

MS m/z: (ES): MH⁺=1151.4.

¹³C NMR (125 MHz, DMSO)/δ: 177.7, 177.2, 171.0, 166.3, 140.5, 137.9, 135.0, 125.5, 124.7, 118.4, 107.2, 102.3, 95.0, 83.0, 77.4, 76.5, 75.1, 73.7, 73.7, 72.7, 70.6, 69.7, 69.5, 69.4, 68.5, 67.4, 67.3, 65.0, 61.8, 61.6, 58.5, 54.4, 53.2, 49.2, 44.9, 41.9, 41.8, 40.5, 36.2, 36.0, 31.2, 30.8, 29.3, 29.1, 27.5, 26.2, 22.2, 21.4, 21.1, 17.9, 17.8, 14.9, 11.1, 9.1, 7.1

### Example 7

### 4″(S)—N-(3-{2-[3-(3-Carboxy-1-ethyl-4-oxo-1,4-dihydro-quinolin-6-yl)-propoxy]-ethoxy}-propionyl)-azithromycin

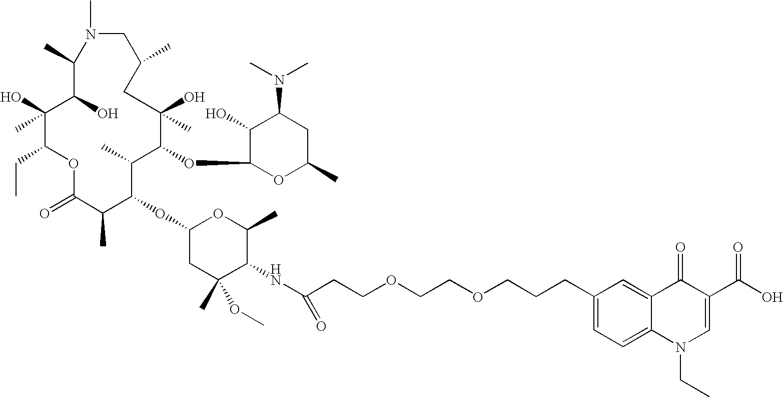

According to the procedure of Example 4 starting from Intermediate 20A (200 mg, 0.27 mmol) and Intermediate 28d (118 mg, 0.30 mmol) the title compound was obtained (250 mg).

MS m/z: (ES): MH⁺=1121.5.

¹³C NMR (125 MHz, DMSO)/δ: 177.6, 177.3, 170.9, 166.3, 148.7, 140.5, 137.6, 135.1, 125.7, 124.8, 118.3, 107.7, 102.7, 94.6, 82.6, 77.7, 76.5, 75.0, 73.8, 72.8, 72.4, 70.7, 69.7, 69.6, 69.5, 68.8, 67.6, 67.2, 64.9, 64.3, 61.6, 57.9, 49.1, 49.1, 44.9, 41.9, 41.7, 40.5, 36.3, 35.8, 34.4, 31.2, 30.8, 30.4, 27.6, 26.2, 22.3, 21.4, 21.4, 21.1, 18.6, 17.9, 15.0, 14.8, 11.1, 9.2, 6.9.

### Example 8

### 4″(S)—N-(3-{2-[3-(3-Carboxy-1-cyclopropyl-4-oxo-1,4-dihydro-quinolin-6-yl)-propoxy]-ethoxy}-propionyl)-azithromycin

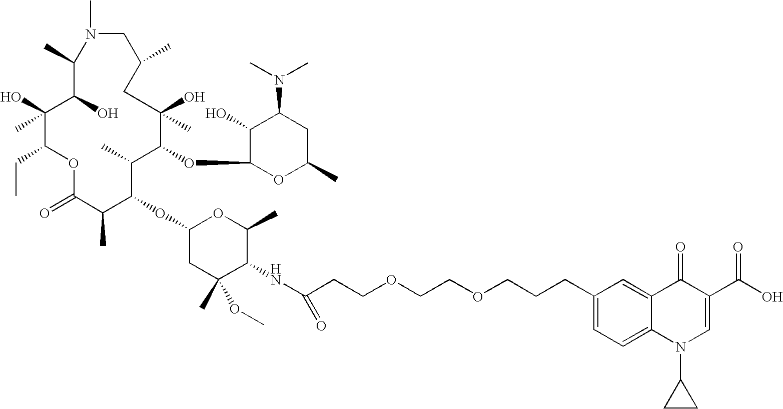

According to the procedure of Example 4 starting from Intermediate 20A (200 mg, 0.27 mmol) and Intermediate 27d (118 mg, 0.30 mmol) the title compound was obtained (170 mg).

MS m/z: (ES): MH⁺=1133.4.

### Example 9

### 4″(R)—N-(3-{2-[3-(3-Carboxy-1-cyclopropyl-4-oxo-1,4-dihydro-quinolin-6-yl)-propoxy]-ethoxy}-propionyl)-azithromycin

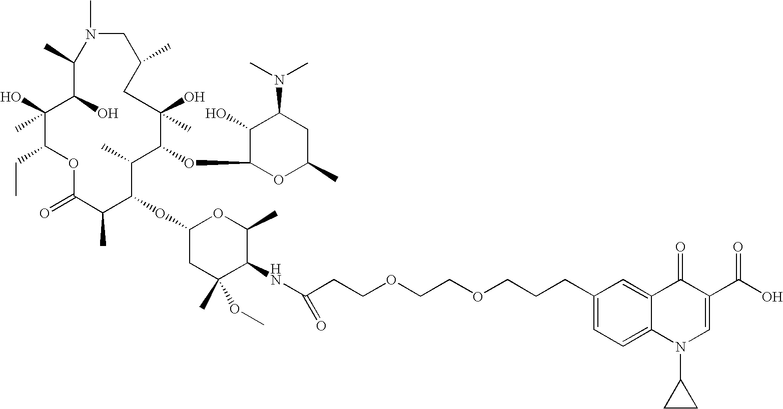

According to the procedure of Example 4 starting from Intermediate 20B (300 mg, 0.40 mmol) and Intermediate 27d (177 mg, 0.44 mmol) the title compound was obtained (250 mg).

MS m/z: (ES): MH⁺=1133.3.

### Example 10

Using a standard broth dilution method in microtitre, compounds were tested for antibacterial activity.

The MIC (μg/ml) of test compounds against various organisms was determined including: Staphylococcus aureus, Streptococcus pneumoniae, Streptococcus pyogenes, Enterococcus faecalis, Haemophylus influenzae, Moraxella catarrhalis.

The compounds in the above examples gave minimum inhibitory concentrations (MICs) equal or less than 1 microgram per millilitre against erythromycin-sensitive and erythromycin-resistant strains of Streptococcus pneumoniae and Streptococcus pyogenes.

Examples 3, and 6-9 have an MIC≦0.5 μg/ml against sensitive Staphylococcus aureus, and examples 3, 7 and 8 have MIC≦0.125 μg/ml against Enterococcus faecalis. Example 3 has MIC≦0.125 μg/ml against Haemophylus influenzae.

Examples 2, 3, 4, and 6-9 have an MIC≦2 μg/ml against Moraxella catarrhalis.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A compound of general formula (I) 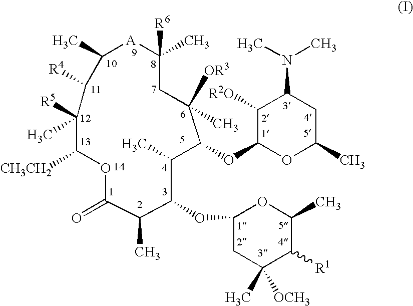
wherein
A is a bivalent radical selected from —C(O)NH—, —NHC(O)—, —N(R⁷)—CH₂— and —CH₂—N(R⁷)—;
R¹ is —NHC(O)(CH₂)_{d}XR⁸;
R² is hydrogen;
R³ is hydrogen, C₁₋₄alkyl, or C₂₋₆alkenyl optionally substituted by 9 to 10 membered fused bicyclic heteroaryl;
R⁴ is hydroxy, C₂₋₆alkenyloxy optionally substituted by 9 to 10 membered fused bicyclic heteroaryl, or C₁₋₆alkoxy optionally substituted by C₁₋₆alkoxy or —O(CH₂)ₑNR⁷R⁹,
R⁵ is hydroxy, or
R⁴ and R⁵ taken together with the intervening atoms form a cyclic group having the following structure: 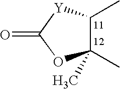
wherein Y is a bivalent radical selected from —CH₂—, —CH(CN)—, —O—, —N(R¹⁰)— and —CH(SR¹⁰)—, with proviso that when A is —NHC(O)—, —N(R⁷)—CH₂— or —CH₂—N(R⁷)—, Y is —O—;
R⁶ is hydrogen or fluorine;
R⁷ is hydrogen or C₁₋₆alkyl;
R⁸ is a heterocyclic group having the following structure: 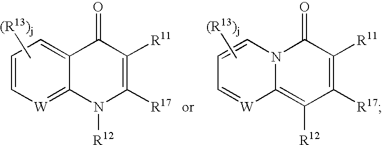
R⁹ is hydrogen or C₁₋₆alkyl;
R¹⁰ is hydrogen or C₁₋₄alkyl substituted by a group selected from optionally substituted phenyl, optionally substituted 5 or 6 membered heteroaryl and optionally substituted 9 to 10 membered fused bicyclic heteroaryl;
R¹¹ is hydrogen, —C(O)OR¹⁴, —C(O)NHR¹⁴, —C(O)CH₂NO₂ or —C(O)CH₂SO₂R⁷
R¹² is hydrogen; C₁₋₄alkyl optionally substituted by hydroxy, cyano, C₁₋₄alkoxyNH₂, —NH(C₁₋₄alkyl) or —N(C₁₋₄alkyl)₂; C₂₋₄alkenyl optionally substituted by hydroxy, cyano, C₁₋₄alkoxy NH₂, —NH(C₁₋₄alkyl) or —N(C₁₋₄alkyl)₂; C₁₋₄alkoxy; C₃₋₇cycloalkyl; —NH₂;
—NH(C₁₋₄alkyl); —N(C₁₋₄alkyl)₂; (C₁₋₄alkyl)OC(O)N(C₁₋₄alkyl); or optionally substituted phenyl or benzyl;
R¹³ is halogen, C₁₋₄alkyl, C₁₋₄thioalkyl, C₁₋₄alkoxy, —NH₂, —NH(C₁₋₄alkyl) or —N(C₁₋₄alkyl)₂;
R¹⁴ is hydrogen or C₁₋₆alkyl optionally substituted by up to three groups independently selected from halogen, C₁₋₁₄alkoxy, —OC(O)C₁₋₆alkyl and —OC(O)OC₁₋₆alkyl; —(CH₂)_{q}heterocyclyl, —(CH₂)_{q}heteroaryl, —(CH₂)_{q}aryl, or —(CH₂)_{q}C₃₋₇cycloalkyl;
R¹⁵ is hydrogen, C₁₋₄alkyl, C₃₋₇cycloalkyl, optionally substituted phenyl or benzyl, acetyl or benzoyl;
R¹⁶ is hydrogen or R¹³, or R¹⁶ and R¹² are linked to form the bivalent radical —O(CH₂)₂—, —(CH₂)ₜ—, —NR⁷(CH₂)ₐ—, —OCH₂NR⁷—, —SCH₂NR⁷—, —CH₂NR⁷CH₂—, —CH₂OCH₂—, —CH₂SCH₂— or —(CH₂)ₐNR⁷—;
R¹⁷ is hydrogen, or R¹⁷ and R¹² are linked to form the bivalent radical selected from the group —S(CH₂)_{b}—, —NR⁷(CH₂)_{b}—, and —O(CH₂)_{b}—;
R¹⁸ is C₁₋₈alkyl, C₂₋₆alkenyl or C₂₋₆alkynyl;
X is —U(CH₂)ᵥB(CH₂)ᵥD-, —U(CH₂)ᵥB—R¹⁸—, —U(CH₂)ᵥB(CH₂)ᵥD(CH₂)ᵥE-, —U(CH₂)ᵥB(CH₂)ᵥD-R¹⁸—
or a group selected from: 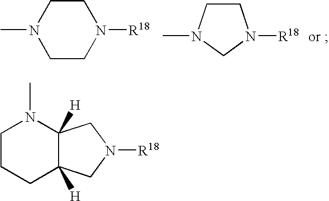
U, B, D and E are independently divalent radicals selected from —N(R¹⁵)—, —O—, —S(O)_{z}—, —N(R¹⁵)C(O)—, —C(O)N(R¹⁵)— and —N[C(O)R¹⁵]—;
W is —C(R¹⁶)— or —N—;
a is 1 or 2
b is an integer from 1 to 3
d is 0 or an integer from 1 to 5;
e is an integer from 2 to 4;
j and z are each independently integers from 0 to 2;
q is an integer from 0 to 4
t is 2 or 3;
v is an integer from 1 to 8;
and pharmaceutically acceptable derivatives thereof.

2. A compound according to claim 1 wherein A is —N(R⁷)—CH₂—.

3. A compound according to claim 1 wherein X is —O(CH₂)₂O(CH)₂N—, —N(CH₂)O(CH₂)₂O(CH₂)N— or —O(CH₂)₂O(CH₂)₃—.

4. A compound according to claim 1 wherein d is 2 or 3.

5. The compound of claim 1 wherein
A is —N(R⁷)—CH₂— or —C(O)—;
R¹ is —NHC(O)(CH₂)_{d}R⁸;
R² is hydrogen;
R³ is hydrogen, C₁₋₄alkyl, or C₂₋₆alkenyl;
R⁴ and R⁵ are hydroxy; and
R⁷ is hydrogen or C₁₋₆alkyl,
and pharmaceutically acceptable derivatives thereof.

6. The compound of claim 1 wherein
R⁸ is a heterocyclic group having the following structure: 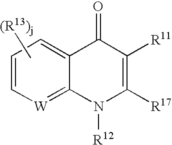
wherein the heterocyclic is linked in the 6 or 7 position,
W is —CH— or —N—;
R¹¹ is hydrogen, —C(O)OR¹⁴, —C(O)NHR¹⁴, —C(O)CH₂NO₂ or —C(O)CH₂SO₂R⁷;
R¹² is hydrogen; C₁₋₄alkyl optionally substituted by C₁₋₄alkoxy; NH₂; C₃₋₇ cycloalkyl or —N(C₁₋₄alkyl)₂;
R¹³ is halogen, C₁₋₄alkoxy, or —NH₂;
R¹⁴ is hydrogen or C₁₋₆alkyl; and
R¹⁷ is hydrogen,
and pharmaceutically acceptable derivatives thereof.

7. The compound of claim 1 wherein
X is —U(CH₂)ᵥB(CH₂)ᵥD-, —U(CH₂)ᵥB—R¹⁸—, —U(CH₂)ᵥB(CH₂)ᵥD(CH₂)ᵥE-, or —U(CH₂)ᵥB(CH₂)ᵥD-R¹⁸—;
U, B, D and E are independently divalent radicals selected from —N(R¹⁵)—, —O—, —S(O)_{z}—;
R¹⁵ is hydrogen or C₁₋₄alkyl; and
R¹⁸ is C₁₋₈alkyl, C₂₋₆alkenyl or C₂₋₆alkynyl,
and pharmaceutically acceptable derivatives thereof.

8. The compound of claim 1 wherein R¹¹ is —C(O)OH and pharmaceutically acceptable derivatives thereof.

9. The compound according to claim 1 wherein X is —O(CH₂)ᵥO(CH₂)ᵥN(R³⁰)— or —O(CH₂)ᵥOR³³—; R³⁰ is H or CH₃, and R³³ is C₁₋₈alkyl, C₂₋₆alkenyl or C₂₋₆alkynyl.

10. A compound according to claim 1 wherein R⁸ is a heterocyclic group of the following formula: 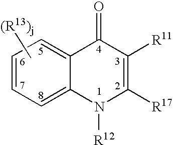
wherein the heterocyclic is linked in the 6 or 7 position and j, R¹¹, R¹², R¹³ and R¹⁷ are as defined in claim 1.

11. The compound of claim 10, wherein
R¹¹ is hydrogen, —C(O)OH, or —C(O)NH₂;
R¹² is hydrogen, C₁₋₄alkyl optionally substituted by hydroxy cyano, or NH₂; NH₂; C₁₋₄alkoxy, or C₃₋₇cycloalkyl;
R¹³ is halogen; and
R¹⁷ is hydrogen,
and pharmaceutically acceptable derivatives thereof.

12. A compound according to claim 1 as defined in any one of Examples 1 to 9, or a pharmaceutically acceptable derivative thereof.

13. A compound selected from:
4″(R)—N-(3-{2-[2-(3-Carboxy-7-chloro-1-cyclopropyl-4-oxo-1,4-dihydro-quinolin-6-ylamino)-ethoxy]-ethoxy}-propionyl}-azithromycin;
4″(S)—N-(3-{2-[2-(3-Carboxy-7-chloro-1-cyclopropyl-4-oxo-1,4-dihydro-quinolin-6-ylamino)-ethoxy]-ethoxy}-propionyl}-azithromycin;
4″(R)—N-(3-{2-[3-(3-Carboxy-1-ethyl-4-oxo-1,4-dihydro-quinolin-6-yl)-propoxy]-ethoxy}-propionyl)-azithromycin;
4″-(R)—N-(3-{2-[3-(3-Carboxy-1-dimethylamino-4-oxo-1,4-dihydro-quinolin-6-yl)-propoxy]-ethoxy}-propionyl)-azithromycin;
4″-(R)—N-(3-{2-[3-(3-Carboxy-1-(2-methoxy-ethyl-4-oxo-1,4-dihydro-quinolin-6-yl)-propoxy]-ethoxy}-propionyl)-azithromycin;
4″ (S)—N-(3-{2-[3-(3-Carboxy-1-ethyl-4-oxo-1,4-dihydro-quinolin-6-yl)-propoxy]-ethoxy}-propionyl)-azithromycin;
4″ (S)—N-(3-{2-[3-(3-Carboxy-1-cyclopropyl-4-oxo-1,4-dihydro-quinolin-6-yl)-propoxy]-ethoxy}-propionyl)-azithromycin; and
4″ (R)—N-(3-{2-[3-(3-Carboxy-1-cyclopropyl-4-oxo-1,4-dihydro-quinolin-6-yl)-propoxy]-ethoxy}-propionyl)-azithromycin
or a pharmaceutically acceptable derivative thereof.

14. A process for the preparation of a compound as claimed in claim 1 which comprises:
a) reacting a compound of formula (II) 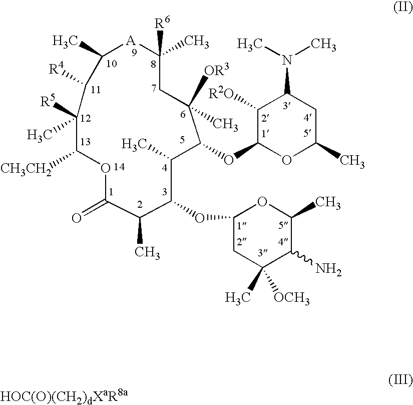
with a suitable activated derivative of the acid (III), wherein X^{a} and R^{8a} are X and R⁸ as defined in claim 1 or groups convertible to X and R⁸ to produce a compound of claim 1 wherein d is an integer from 1 to 5;
b) reacting a compound of formula (II) with an isocyanate OCNX^{a}R^{8a} to produce a compound of claim 1 wherein d is 0 and U is —NH—;
c) reacting a compound of formula (II) with a carbamoyl chloride ClC(O)N(R¹⁵)X^{a}R^{8a} to produce a compound of claim 1 wherein d is 0 and U is —N(R¹⁵)—;
d) reacting a compound of formula (IV) 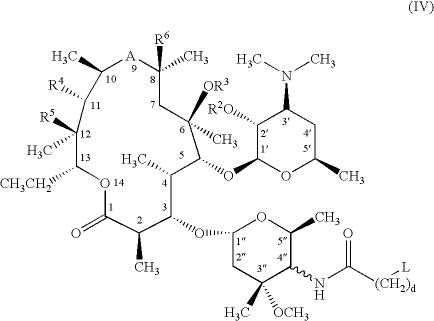
with a compound of formula X^{a}R^{8a} (V), wherein R^{8a} and X^{a} is R⁸ and X as defined in claim 1 or a group convertible to R⁸ and X, L is suitable leaving group, to produce a compound of claim 1 wherein U is a group selected from —N(R¹⁵)—, —O— and —S—; or
e) converting one compound of formula (I) into another compound of formula (I); and thereafter, if required, converting the resultant compound of formula (I) into a pharmaceutically acceptable derivative thereof.

15-17. (canceled)

18. A method for the treatment of the human or non-human animal body to combat microbial infection comprising administration to a body in need of such treatment of an effective amount of a compound as claimed in claim 1.

19. A pharmaceutical composition comprising at least one compound as claimed in claim 1 in association with a pharmaceutically acceptable excipient, diluent and/or carrier.
